(19) United States
(12) Patent Application Publication
(10) Pub. No.: US 2009/0049890 A1
(43) Pub. Date: Feb. 26, 2009

(54) MULTI-MODULED NANOPARTICLE-STRUCTURED SENSING ARRAY AND PATTERN RECOGNITION DEVICE FOR DETECTION OF ACETONE IN BREATH

(75) Inventors: Chuan-Jian ZHONG, Endwell, NY (US); Lingyan WANG, Binghamton, NY (US); Susan LU, Vestal, NY (US); Xiajing SHI, Binghamton, NY (US); Weibing HAO, Vestal, NY (US); Jin LUO, Vestal, NY (US)

Correspondence Address: NIXON PEABODY LLP - PATENT GROUP, 1100 CLINTON SQUARE, ROCHESTER, NY, 14604, US

(73) Assignee: Research Foundation of State University of New York, Binghamton, NY (US)

(21) Appl. No.: 12/104,984

(22) Filed: Apr. 17, 2008

Related U.S. Application Data
(60) Provisional application No. 60/912,618, filed on Apr. 18, 2007.

(51) Int. Cl. G01N 33/497

(52) U.S. Cl. 73/23.3

(57) ABSTRACT

The present invention is directed toward a multi-moduled nanoparticle-structured sensing array and pattern recognition device for detection of acetone in breath.

This application claims the benefit of U.S. Provisional Patent Application Ser. No. 60/912,618, filed Apr. 18, 2007, which is hereby incorporated by reference in its entirety.

The subject matter of this application was made with support from the United States Government under National Science Foundation, Grant No. CHE 0349040. The U.S. Government has certain rights.

### FIELD OF THE INVENTION

The present invention relates to a multi-moduled nanoparticle-structured sensing array and pattern recognition device for detection of acetone in breath.

### BACKGROUND OF THE INVENTION

There are two main kinds of diabetes. Type 1 (juvenile diabetes or insulin-dependent diabetes) is usually first diagnosed in children or teenagers. In this form of diabetes, the beta cells of the pancreas no longer make insulin, because the body's immune system has attacked and destroyed them. Without insulin, sugar builds up in the blood and can damage internal organs, the nervous system and blood vessels. Type 2 (noninsulin-dependent diabetes) is the most common form of diabetes. People can develop type 2 diabetes at any age—even during childhood. This form of diabetes usually begins with insulin resistance, a condition in which fat, muscle, and liver cells do not use insulin properly.

According to American Diabetes Association, there are 20.8 million people have diabetes in the United States, of which an estimated 14.6 million have been diagnosed with diabetes, but unfortunately, 6.2 million people are unaware that they have the disease. The World Health Organization estimates that there are 177 million people worldwide who have diabetes in 2000, which increases to at least 300 million by 2025.

The frequent monitoring of blood glucose levels in individuals with diabetes mellitus has become a major burden for the patients. Due to the need for multiple daily measurements, the current invasive blood test kits are both a painful experience and a cost burden for the patients. The cost of diabetes testing supplies alone can easily exceed $1,200 a year for someone who tests their blood sugar three times a day. As a result, a non-invasive glucose device measurement device is extremely desirable. However, such a cost-effective measurement device is not available on the market so far. Patents on the non-invasive glucose measurement device exist, e.g., near infrared radiation and spectroscopic absorption techniques, for example see U.S. Pat. No. 5,070,874. There are also other similar devices for monitoring glucose levels using both reflectance and transmission spectroscopic techniques. However, there are several problems with the practical applications of these types of devices, including overlap of the spectrum of glucose with other blood chemicals and the difficulty of discriminating between metabolized and excreted sugars.

The formation of acetone in the blood happens when the body uses fat instead of glucose for generating energy. This occurrence usually means that the cells do not have enough insulin, or cannot use the insulin available to facilitate the use of glucose for generating energy. The acetone produced can either pass through the body into the urine or go through breath to generate smells (acetone breath). The quantitative detection of acetone levels in human breath is therefore considered to be an important diagnostic and monitoring tool for the diabetes.

The interparticle physical or chemical properties of molecularly-capped nanoparticles have been explored for chemical sensing in a number of significant ways. See Templeton, A., et al., Acc. Chem. Res., 33: 27 (2000); Daniel, M., et al., Chem. Rev., 104: 293 (2004); Zhong, C., et al., Nanoparticle Assemblies and Superstructure Ed. by N. Kotov, Marcel Decker Publishers (2005); Wohltjen, H., et al., Anal Chem., 70: 2856 (1998); Evans, S., et al., J. Mater. Chem., 10: 183 (2000); Severin, E., et al., Anal. Chem., 72: 2008 (2000); Shinar, R., et al., Anal Chem., 72: 5981 (2000); Han, L., et al., Anal. Chem., 73: 4441 (2001); Houser, E., et al., Talanta, 54: 469 (2001); Zamborini, F., et al., J. Am. Chem. Soc., 124: 8958 (2002); Zamborini F., et al., Anal. Chim. Acta, 496: 3 (2003); Cai, Q., et al., Anal. Chem., 74: 3533 (2002); Grate, J., et al., Anal Chem., 75: 1868 (2003); Grate, J., et al., Anal. Chem., 75: 1868 (2003); Joseph, Y., et al., J. Phys. Chem. B, 107: 7406 (2003); Joseph, Y., et al., Faraday Discuss., 125: 77 (2004); and Joseph, Y., et al., Sens. Actuators B., 98: 188 (2004).

In the past several years, there has been an increase in research and development directed toward the detection of acetone in human breath. Some of the major problems encountered include inadequate detection limit, low selectivity, lack of portability, high rate of false alarms, and high cost of instrumentation. These problems constitute major obstacles to the development, marketing, and commercialization of breath acetone sensors for diagnostics of diabetes.

The present invention is directed to overcoming these and other deficiencies in the art.

### SUMMARY OF THE INVENTION

One aspect of the present invention is directed toward a detector for acetone comprising a sensing platform comprising thin film assemblies of metal or alloy core, ligand-capped nanoparticles and molecular linkers connecting the nanoparticles. This detector includes a plurality of transducers mounted on the sensing platforms. This detector also includes an artificial neural network operably linked to a voltage source and the plurality of transducers and designed to recognize contact of acetone with the sensing platform.

Another aspect is directed to a method of detecting acetone in a fluid comprising providing a fluid and contacting the fluid with the detector of the present invention under conditions effective to detect acetone in the fluid.

In addition to the low concentration level of acetone in breath (50-60 ppb), the presence of water, CO₂, and other gases in breath poses technical complexity for the sensor design and elimination of false alarm. The present invention focuses on the development of Portable Sensor Array (PSA) technology coupled with nanostructured sensing materials and an intelligent pattern recognition engine in a handheld device which can detect the level of acetone in human breath accurately, rapidly, and without false alarming. This product will integrate sensing array nanomaterials, pattern recognition, and compact electronic hardware with the desired detection limit (˜10 ppb) and response speed.

The core product of the technology is a family of PSA devices which can be utilized for the detection of volatile and toxic gases in the environment. The strengths of the PSA device for detecting acetone in human breath for diagnostics of diabetes are the capabilities of the device in terms of the following six important design criteria: (1) the ability to respond to acetone with high sensitivity and low detection limit, (2) the ability to differentiate acetone from other chemicals in the breath with high selectivity to minimize false alarms, (3) rapid response time, (4) device portability, (5) non-invasive detection mode, and (6) cost-effectiveness of the device. Currently, a variety of transducers are available commercially or in research laboratories which can detect volatile organic compounds (VOCs), e.g., ion mobility spectrometers, mass spectrometers, antibody-based technology with optical reporters, gas chromatography and mass spectroscopy, fluorescence-based sensor array, etc. See Schmid, G., Adv. Eng. Mater., 3: 737 (2001); Shipway, A., et al., Chem Phys Chem., 1:18 (2000); Zhong, C., et al., Adv. Mater., 13: 1507 (2001); Wohltjen, H., et al., Anal Chem., 70: 2856 (1998); and Han, L., et al., Anal. Chem., 73: 4441 (2001), which are hereby incorporated by reference in their entirety. The sensitivity, selectivity, and response speed of some systems, especially in monitoring applications, are limited. Most commercial chemiresistor-type gas sensors use semiconductor materials (SnO₂), because they have relatively high sensitivity and simple electronics. The main drawbacks include the lack of selectivity, poor long-term stability, humidity dependence, and high temperature (>300° C.) requirement. Despite many innovations, the complex backgrounds and low concentration in practical applications make the detection an extremely challenging task.

The foundation of the present invention couples nanostructured sensing arrays with chemiresistive (e.g., interdigitated microelectrode (IME)) or piezoelectric (e.g., quartz-crystal microbalance (QCM)) transducer sensing platforms. See Han, L., et al., Anal Chem., 73: 4441 (2001), which is hereby incorporated by reference in its entirety. The detection mechanism is based on the vapor-nanostructure interactions which induce changes in electronic conductivity or in mass loading with unique response signatures which can be identified by pattern recognition technique. The electronic conduction and framework affinity display electronic or mass responses that are highly sensitive due to fine-tunability of size, shape, composition, and spatial properties, large surface area-to-volume ratio, multidentate ligating specificity, and molecularly-defined nanoporosity. See Schmid, G., Adv. Eng. Mater., 3: 737 (2001); Shipway, A., et al., Chem Phys Chem., 1:18 (2000); Zhong, C., et al., Adv. Mater., 13: 1507 (2001); Wohltjen, H., et al., Anal. Chem., 70: 2856 (1998); Han, L., et al., Anal Chem., 73: 4441 (2001); Zamborini, F., et al., J. Am. Chem. Soc., 124: 8958 (2002); Dickert, F., et al., Ber. Bunsen, Phys. Chem., 100: 1312 (1996); and Zheng, W., et al., Anal Chem., 72: 2190 (2000), which are hereby incorporated by reference in their entirety.

The array system includes sensing nanomaterials, transducers, microelectronics, microprocessor, battery-based power supply, and software for data processing and pattern recognition. The coupling of the molecularly-mediated thin film assemblies of nanoparticles and the pattern-recognition in an integrated chip device constitutes an important strength leading to unprecedented enhancement in sensitivity, selectivity, detection limit, and response time. See Han, L., et al., Anal. Chem., 73: 4441 (2001), which is hereby incorporated by reference in its entirety. In addition to the viability of charging a single electron on a single nanoparticle or hopping/tunneling electrons in a collective ensemble of nanoparticles as highly sensitive materials, there are other important technical attributes, including enrichment of ligands and voids in the high surface area-to-volume ratio microenvironment, non-covalent character such as hydrogen-bonding, coordination and van der Waals sites, and chemically-active nanocrystal catalytic sites for tuning selectivity. See Han, L., et al., Anal Chem., 73: 4441 (2001); Zamborini, F., et al., J. Am. Chem. Soc., 124: 8958 (2002); and Zheng, W., et al., Anal Chem., 72: 2190 (2000), which are hereby incorporated by reference in their entirety. These technical attributes should address some of the major weaknesses in existing sensor technology, including high detection limit, limited selectivity, slow response, lack of portability, and high cost.

### BRIEF DESCRIPTION OF THE DRAWINGS

FIGS. 1A-B are schematic drawings of two types of the core-shell nanoparticle thin-film assemblies derived from the one-step route. FIG. 1A shows ligand-capped gold nanoparticles linked by 1,9-nonanedithiolate (NDT). FIG. 1B shows ligand-capped gold nanoparticles linked by 11-mercaptoundecanoic acid (MUA).

FIG. 2 is a schematic drawing of a chemiresistor coated with thin film assemblies of Au and AuAg nanoparticles mediated by HS—(CH₂)ₙ—SH and HOOC—(CH₂)ₙ—COOH of different chain lengths in a sensor array.

FIG. 3 is a schematic drawing of the Input Layer, Hidden Layers, and Output Layers of an Artificial Neural Network.

FIGS. 4A-C show interdigitated microelectrodes (IME). FIG. 3A is an IME; FIG. 3B is ADT-Au thin film on IME (n=10); FIG. 3C is DCA-AuAg thin film on IME (n=16).

FIG. 5A is a graph showing the initial Resistance of thin film assemblies of gold nanoparticles mediated by ADT (HS—(CH₂)ₙ—SH) of different chain length, where n=number of methylene units in alkyl chain. A fitting result using eqns. 1-2 (Rᵢ=5130.9exp[−31.2/(1.5+0.13 n)]) is shown. FIG. 5B is a graph showing the Activation Energy vs. different chain length n. (linear regression: Eₐ=0.0040 n+0.0489).

FIG. 6A shows a plot of the sensor response profile for ADT-Au films to Hx vapor and FIG. 6B shows a graph of sensitivity to Hx vapor for ADT-Au films. ADT-Au films: n=3, 5, 8, 9, 10 linked thin films of Au nanoparticles.

FIG. 7 shows a graph of response sensitivities (S) of a sensor array of ADT-Au thin films with different chain length to vapors of Tl (A,), Bz (B,) and Hx (C,). Fitting results: S (S₀, a, b)=S₀+a exp(b n); Hx: (2.71×10⁻⁵, 5.44×10⁻¹⁰, 1.16); Bz: (6.38×10⁻⁵, 6.65×10⁻¹¹, 1.39); Tl: (1.36×10⁻⁴, 2.08×10⁻⁸, 0.93).

FIG. 8A shows a plot of the sensor response profile for DCA-AuAg films to Hx vapor and FIG. 8B shows a graph of sensitivity to Hx vapor for DCA-AuAg films. DCA-AuAg films: n=10, 12, 13, 14, 16, and 18 linked thin films of AuAg nanoparticles.

FIG. 9 shows a graph of response sensitivities (S) of a sensor array of DCA-AuAg thin films with different chain length to vapors of Tl (A), Bz (B) and Hx (C) vapor. Fitting results: S (a, b, c)=a/(1+exp(−(n−c)/b)); Hx: (3.46×10⁻⁴, 1.05, 11.97); Bz: (1.09×10⁻³, 1.11, 11.65); Tl: (1.09×10⁻³, 1.11, 11.65).

FIG. 10 shows a graph of response sensitivities (S) of ADT-Au and DCA-AuAg thin films with different chain length to vapors of Tl (3), Bz (2) and Hx (1). Fitting results (Sigmoidal): S (a, b, c, S₀)=S₀+a/(1+(n/c)^{b}); Hx: (3.38, −9.52, 12.22, 0.27); Bz: (3.47, −10.46, 11.84, 0.64); Tl: (9.58, −10.49, 11.92, 1.49).

FIGS. 11A-C are schematic drawings, illustrating the interparticle mediating/templating structures derived by manipulating the relative length differences of the interparticle linking molecule X—(CH₂)ₙ—X and the capping molecule S—(CH₂)ₘCH₃, with m=9.

FIG. 12 is a graph showing a scatter plot in Dₘₐₓ−Dₘᵢₙ plane for sensor arrays based on ADT-Au (n=3, 5, 8, 9, 10) (circle) and DCA-AuAg (n=10, 12, 13, 14, 16, 18) (hexagon) films.

FIGS. 13A-C show Principal Component Analysis (PCA) score plots in the PC1-PC2 plane for three sensor arrays: ADT-Au (n=3 and 5) and DCA-AuAg (n=12 and 13) (FIG. 13A); ADT-Au (n=3 and 5) and DCA-AuAg (n=16 and 18) (FIG. 13B); and ADT-Au (n=9 and 10) and DCA-AuAg (n=16 and 18) (FIG. 13C). Vapor Tl (triangle), Bz (square) and Hx (circle).

FIG. 14 is a schematic drawing illustrating film assembly of nanoparticles (MUA-Auₙₘ) as sensing materials.

FIG. 15A shows a plot of the sensor response profile for responses to acetone on a 10-channel array of different design parameters (see FIG. 15C) coated with MUA-Auₙₘ film. The inset shows the graph of acetone sensitivity. FIG. 15B shows a comparison plot of sensor response profiles for channels 1 and 6 in detection of acetone (AC), water (H₂O), and their mixture (AC-H₂O). FIG. 15C is a table of design parameters: FW: finger width; FS: finger spacing, for the 10-channel array of FIG. 15A.

FIG. 16 is a schematic drawing illustrating the design of a prototype sensor array device.

FIG. 17 shows a Principal Component Analysis (PCA) score plot in the PC1-PC2 plane for mixture, water, and acetone. PC1: 97.3%; PC2: 2.5%

FIG. 18A shows a plot of the training curve (performance: 2.62e-009, goal is 0); FIGS. 18B-C show plots of the BPN output (FIG. 18B) and target output (FIG. 18C) for water (X Axis: 9-10) and mixture (X Axis: 1-8). Recognition rate: 100%.

### DETAILED DESCRIPTION OF THE INVENTION

One aspect of the present invention is directed toward a detector for acetone comprising a sensing platform comprising thin film assemblies of metal or alloy core, ligand-capped nanoparticles and molecular linkers connecting the nanoparticles. This detector includes a plurality of transducers mounted on the sensing platforms. This detector also includes an artificial neural network operably linked to a voltage source and the plurality of transducers and designed to recognize contact of acetone with the sensing platform. The transducers may be chemiresistive, e.g. interdigitated microelectrodes, or piezoelectric, e.g. quartz-crystal microbalances. The detector may also include a micro controller and or a circuit board operably linked to the transducers.

The molecular linkers may be α,ω-alkyldithiols, α,ω-dicarboxylic acids, mercaptocarboxylic acids, or combinations thereof. The α,ω-alkyldithiol may be HS—(CH₂)ₙ—SH, with n being 3-10. The α,ω-dicarboxylic acid may be HO₂C—(CH₂)ₙ—CO₂H, with n being 2 to 16. The mercaptocarboxylic acids is HS—(CH₂)ₙ—CO₂H, with n being 2 to 18.

The detector may comprise a plurality of different sensing platforms. The different sensing platforms differ with regard to the nanoparticle capping ligands, the nanoparticle cores, the molecular linkers, and/or film thickness. The nanoparticle cores and capping ligands may differ by size or material. The molecular linkers may differ by length or chemical content.

The neural network may be trained to recognize contact of acetone with the sensing platform or may be trained to distinguish contact of acetone with the sensing platform from contact of other agents with the sensing platform. The neural network may be trained to quantitate acetone concentration contacting the sensing platform.

An array of chemiresistive sensors in which each sensor surface is coated with a different nanostructure has been demonstrated for detection of VOCs. Since each sensor element may respond to VOCs differently, the responses of a sensor array to a certain VOC will show its unique profile or pattern, which can be utilized for identification of the vapors. Hence, the construction of sensor array system involves the rational selection of the sensing nanomaterials and the pattern recognition of sensing signals which play important roles in sensor development for accurately detecting the targeted analytes. Sensing nanomaterials selection is actually a feature space optimization problem for the pattern recognition process, in which the optimal sensors are chosen so that the feature space from the sensor responses for different analytes can be well separated. The pattern recognition of a sensor array involves a process of detecting sample analytes by recognizing certain patterns in the feature space of the sensor array response signals. Although many pattern recognition approaches have been developed, such as statistical methods, neural networks, and fuzzy inference systems, little attention has been given to the optimization of the inputs to pattern classifiers. See Aleixandre, M., et al., Sensors and Actuators B 103: 122-128 (2004); Pardo, M., et al., IEEE Sensors Journal, Vol. 4, No. 3, (2004); Roncaglia, A., et al., IEEE Sensors Journal, Vol. 4, No. 2, (2004); Penza, M., et al., Sensors and Actuators B 89: 269-284 (2003); Llobet, E., et al., Sensors and Actuators B 83: 238-244 (2002); and Jurs, P., et al., Chem. Rev., 100: 2649-2678 (2000), which are hereby incorporated by reference in there entirety. A hybrid method which couples multi-module method with artificial neural networks (ANNs) for the optimization—Optimized Multi-module ANN Classifier (OMAC) has been developed. See Han, L., et al., Sensors and Actuators, B., 106: 431-441 (2005) which is hereby incorporated by reference in its entirety. This method is developed to target sensor arrays for detecting multiple VOCs.

The Optimized Multi-module ANN Classifier (OMAC) method is based on applying ANNs to nanostructured sensor arrays for the detection of multiple VOCs. The OMAC method combines multiple-module ANNs with sensor array optimization technology and pattern recognition for more effective nanostructured array sensing. See Han, L., et al., Sensors and Actuators, B., 106: 431-441 (2005) which is hereby incorporated by reference in its entirety. In this approach, each ANN module is dedicated to a sub group/class of VOCs. More importantly, each ANN module has its own optimum inputs. Important aspects include: (1) a systematic approach for optimum selection of sensor nanomaterials; (2) pattern recognition techniques using multi-module ANNs, supported by Cluster Analysis (CA), and (3) input optimization of the sensor responses to each dedicated ANN module.

The nanoparticle capping ligand may be alkanethiols, alkyl amines, alkyl alcohols, alkanoic acids, or mixtures thereof. The nanoparticle capping ligand may be decanethiol.

The core material of the nanoparticles may be gold, silver, platinum, iron oxide, gold-silver alloy, gold-platinum alloy, gold-copper alloy, or mixtures thereof.

While solvent evaporation is a straightforward method of nanoparticle thin film preparation, it faces serious difficulties concerning structural manipulation and stability. Upon vapor sorption, extensive structural rearrangement of the deposited particles easily occur due to weak hydrophobic interactions resulting in altering the thin film's electronic properties. The introduction of molecular linkages permits structural controllability at the molecular level, which is evidenced by recent progress in the development of nanoparticle assembling strategies such as DNA-linking, place-exchange, and stepwise layer-by-layer construction. An effective nanoconstruction route termed one-step exchange-crosslinking-precipitation route has been developed based on the place-exchange reactivity of core-shell nanoparticles. See Hostetler, M., et al., J. Am. Chem. Soc., 118: 4212 (1996); Ingram, R., et al., J. Am. Chem. Soc., 119: 9175 (1997); Hostetler, M., et al., Langmuir, 15: 3782 (1999); Templeton, A., et al., J. Am. Chem. Soc., 120: 1906-1911 (1998); Templeton, A., et al., J. Am. Chem. Soc., 120: 4845 (1998), which are hereby incorporated by reference in their entirety. This route is applicable for assembling nanoparticle network thin films onto many types of substrate. See Leibowitz, F., et al., Anal Chem., 71: 5076 (1999); Zheng, W, et al., Anal Chem., 72: 2190 (2000); and Han, L., et al., J. Mater. Chem., 11: 1259 (2001), which are hereby incorporated by reference in their entirety.

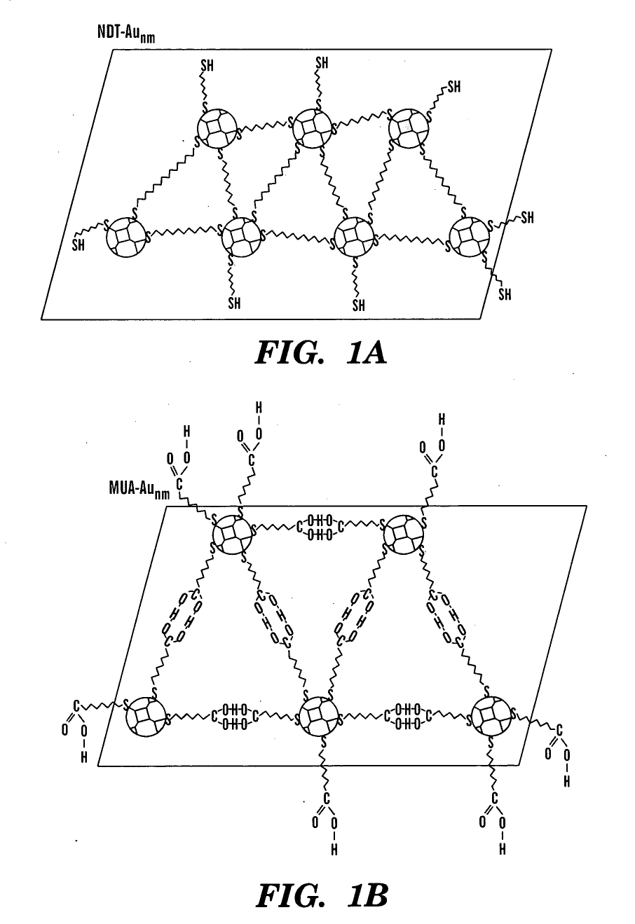

FIG. 1 illustrates two types of the core-shell nanoparticle thin film assemblies derived from a one-step route. The nanostructures involve two different co-functionalized thiol linkages, the covalent bonding at both ends of 1,9-nonanedithiolate and the head-to-head hydrogen-bonding at the terminals of the gold-bound 11-mercaptoundecanoic acid. Because of the simplicity of the film preparation and the possibility for structural tailoring of interparticle spatial and chemical properties, the nanoparticle film assembly offers clear advantages over evaporation-prepared films as chemically-sensitive interfaces. The nanostructured films are electronically conductive depending on core size and molecular linkage properties. See Leibowitz, F., et al., Anal Chem., 71: 5076 (1999); Zheng, W, et al., Anal Chem., 72: 2190 (2000); Han, L., et al., J. Mater. Chem., 11: 1259 (2001); and Han, L., et al., Anal Chem., 73: 4441 (2001), which are hereby incorporated by reference in their entirety. The present invention may utilize electronic resistance responses to interfacial vapor sorption at nanostructured films.

Another aspect is directed to a method of detecting acetone in a fluid comprising providing a fluid and contacting the fluid with the detector of the present invention under conditions effective to detect acetone in the fluid. The specific materials used to form the detector are substantially the same as those described above. The detector may comprise a plurality of different sensing platforms. The fluid may be a gaseous stream which may be a breath stream.

Since the early report on spraying alkanethiolate-protected nanoparticles as metal-insulator-metal ensemble on chemiresistors for vapor sensing, a number of nanoparticle-structured thin films have been studied for chemical sensors. See Wohltjen, H., et al., Anal. Chem., 70: 2856 (1998); Evans, S., et al., J. Mater. Chem., 10: 183 (2000); Severin, E., et al., Anal. Chem., 72: 2008 (2000); Shinar, R., et al., Anal. Chem., 72: 5981 (2000); Han, L., et al., Anal. Chem., 73: 4441 (2001); Houser, E., et al., Talanta, 54: 469 (2001); Zamborini, F., et al., J. Am. Chem. Soc., 124: 8958 (2002); Zamborini F., et al., Anal Chim. Acta, 496: 3 (2003); Cai, Q., et al., Anal. Chem., 74: 3533 (2002); Grate, J., et al., Anal Chem., 75: 1868 (2003); Joseph, Y., et al., J. Phys. Chem. B, 107: 7406 (2003); Joseph, Y., et al., Faraday Discuss., 125: 77 (2004); and Joseph, Y., et al., Sens. Actuators B., 98: 188 (2004), which are hereby incorporated by reference in their entirety. One example is the use of carboxylate-Cu²⁺-carboxylate bridged nanoparticles for vapor sensing via a swelling-induced alteration in chemical nature of electron tunneling. See Zamborini, F., et al., J. Am. Chem. Soc., 124: 8958 (2002) and Zamborini F., et al., Anal. Chim. Acta, 496: 3 (2003), which are hereby incorporated by reference in their entirety. Another example is molecularly-mediated thin film assembly of nanoparticles via covalent bonding or hydrogen-bonding on both chemoresistive and piezoelectric sensors for vapor sensing. See Han, L., et al., Anal Chem., 73: 4441 (2001), which is hereby incorporated by reference in its entirety. Such thin film nanostructures were recently shown to be viable for constructing sensor array materials when the array is coupled to pattern recognition engine. See Han, L., et al., Sens. Actuators B., 106: 431 (2005); Shi, X., et al., Sens. Actuators B., 117: 65 (2006); and Wang, L., et al., Sensors., 6: 667 (2006), which are hereby incorporated by reference in their entirety. Core-shell type nanoparticles, which are broadly defined as nanocrystal core and molecular shell of different matters in close interaction, are intriguing building blocks to sensing array materials because the ability to tune size, composition, functional group and interparticle spatial properties provides effective ways for the enhancement in sensitivity, selectivity, detection limit and response time. See Zhong, C., et al., Adv. Mater., 13: 1507-1511 (2001); Krasteva, N., et al., Sens. Actuators B., 92: 137 (2003); Leopold, M., et al., Faraday Discuss., 125: 63 (2004); Ahn, H., et al., J. Macromol. Sci., Pure Appl. Chem., A42: 1477 (2005); Lavine, B., et al., Anal. Chem., 78: 4137 (2006); Yang, C., et al., Anal. Chim. Acta., 565: 17 (2006); Ibanez, F., et al., Anal Chem., 78: 753 (2006); Pang, P., et al., Sens. Actuators B., 114: 799 (2006); and Franke, M., et al., Small, 2: 36 (2006), which are hereby incorporated by reference in their entirety. This ability stems from several important attributes of nanoparticle assemblies in designing sensing array materials, including the enrichment of ligands or voids in the high surface area-to-volume ratio microenvironment to provide framework and nanoporosity for signal amplifications, the introduction of non-covalent characters such as hydrogen-bonding, coordination, and van der Waals sites through the shell and linker molecules to provide tunable molecular interactions for enhancing selectivity, and the coupling of nanostructures to the chemoresistive or piezoelectric transducers with easy array integration, rapid response and low power-driven capability. The demonstration of ion-gating channels with biomimetic specificity parallels synthetic or biological receptors. See Zheng, W., et al., Anal. Chem., 72: 2190 (2000) and Dickert, F., et al., Ber. Bunsen-Ges. Phys. Chem., 100: 1312 (1996), which are hereby incorporated by reference in their entirety.

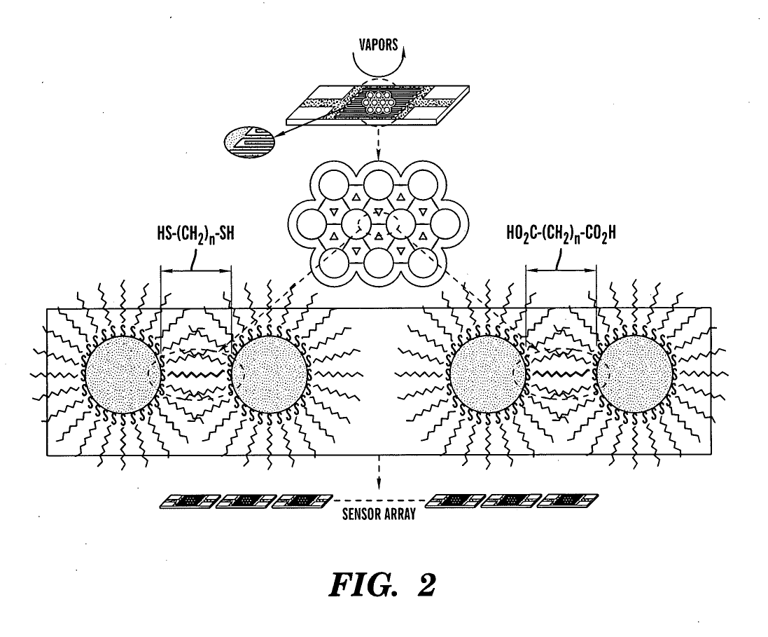

A key element in using the nanostructured thin film materials to design chemoresistive sensor arrays is the correlation between the electronic conductivity and the nanostructure parameters including nanoparticle core radius, interparticle distance, and dielectric constant of interparticle medium. The parameters determine the activation energy in a thermally-activated conduction path. See Hostetler, M., et al., Langmuir, 15: 3782 (1999) and Abeles, B., et al., Adv. Phys., 24: 407 (1975), which are hereby incorporated by reference in their entirety. Little has been reported on such a systematic correlation. Built upon recent successful demonstrations of molecularly-mediated thin film assemblies of monometallic Au and bimetallic AuAg nanoparticles with alkyl dithiols and dicarboxylic acids of different chain lengths, the use of these two types of spatially-controlled sensing nanostructures as sensor array materials for establishing the correlation between sensor responses and interparticle spatial properties are disclosed herein. See Leibowitz, F., et al., Anal Chem., 71: 5076 (1999); Han, L., et al., J. Mater. Chem., 11: 1258 (2001); and Kariuki, N., et al., Chem. Mater., 18: 123 (2006), which are hereby incorporated by reference in their entirety. In these nanostructures, Au or AuAg nanoparticles capped with alkanethiolates are used as building blocks for the molecularly-mediated thin film assembly. It is the capability of thin film assembly by mediator molecules of different length which distinguishes the present invention from other existing approaches to constructing sensing materials. For the thin film assembly of Au nanoparticles mediated by an alkyl dithiol (ADT, i.e., HS—(CH₂)ₙ—SH) (ADT-Au), the interparticle linkage is formed by Au-thiolate bonds at both ends of the dithiol. For the thin film assembly of AuAg nanoparticles mediated by a dicarboxylic acid (DCA, i.e., HOOC—(CH₂)ₙ—COOH) (DCA-AuAg), the interparticle linkage is formed by selective ionic binding between carboxylates of the linker and the Ag sites of the bimetallic nanoparticles. The thin film assemblies derived from mediators of different chain lengths form the basis for the design of sensing array nanomaterials in terms of interparticle spatial properties. See FIG. 2.

While the thin film assemblies of gold and gold-silver alloy nanoparticles using ADTs and DCAs with different alkyl chain length (m, i.e., number of methylenes in the alkyl chain) are different in terms of the binding nature and nanoparticle composition, their common character of the tunable alkyl chain length provides an important means for assessing the correlation between the sensing properties and the interparticle spatial parameters. The sensing properties are demonstrated by testing the thin films assembled on interdigitated microelectrode (IME) for the detection of volatile organic compounds (VOCs). The further coupling of the sensor array data with pattern recognition techniques provides insights into the detailed delineation of the interparticle spatial properties for constructing nanostructured sensing arrays. See Zellers, E., et al., Sens. Actuators B, 12: 123 (1993); Bakken, G., et al., Sens. Actuators B, 79: 1 (2001); Gardner, J, Sens. Actuators B, 4: 109 (1991); Corcoran, P., et al., Sens. Actuators B, 48: 448 (1998), which are hereby incorporated by reference in their entirety.

There are many existing approaches to pattern recognition with sensor arrays, including, Artificial Neural Networks (ANNs), Cluster Analysis (CA), and Principal Component Analysis (PCA) techniques. Among all these methods, ANNs are universally recognized as one of the most effective approaches. However, the current ANNs applications in sensor array only involve single ANN module/architecture, which is very difficult to simultaneously obtain the satisfying correct identification rates for multiple VOCs. The present invention utilizes a more sophisticated multi-module ANNs approach with each module dedicated to a sub group/class of VOCs. Each specific module may have different sensing signals as inputs, which are determined by the results of sensor optimization for each vapor subgroup. The CA technique is used for arranging the vapors to different groups, with each group having one dedicated ANNs module to serve as specific vapor recognizer.

Cluster Analysis (CA) is used to group the vapors for assisting the construction of multi-module ANNs. In this method, the responses from the sensor array are first processed with PCA technique to extract the main distinguishing features for different patterns. The PCA data for different VOCs are then clustered into different classes based on their similarities obtained by cluster analysis techniques. Different separation distance scales make it necessary to group a set of target vapors into different subgroups with each one having a dedicated intelligent classifier, before individual vapor is identified. The objective of this step is to further enhance the correct recognition rate for each individual vapor. The grouping task is accomplished by cluster analysis technique with K-clustering algorithm.

CA is a statistical method for assigning sets of similar items into different groups (clusters) with meaningful structures. There are different algorithms and approaches for clustering. The K-clustering algorithm is one of the most common nonparametric partition-clustering one for exclusive test patterns which attempts to find a K-clustering with minimal MSE. See McQueen, J., Some Methods for Classification and Analysis of Multivariate Observation, Computer and Chemistry, 4: 257-272 (1967), which is hereby incorporated by reference in its entirety. In other words, its goal is to minimize dissimilarity in the items within each cluster while maximizing the value between items in different clusters. It searches for the best set of clusters centroids, and determines the structure of the partition by assigning each input vector to its nearest centroid. The centroid of a cluster is defined as the point whose value are the average values of every point in the current cluster. The distance to the centroids is calculated based on Euclidean distance metric, which is given by Equation (4):

$${Ed}_{ij} = \sqrt{{\sum }_{k = 1}^{n} {( {x}_{ik} - {x}_{jk} )}^{2}} , \qquad ( 4 )$$

where Edᵢⱼ is the Euclidean distance between patterns xᵢ and xⱼ, each with n samples.

Principle Component Analysis is a mathematical method that converts a large number of potentially correlated variables into relatively small number of uncorrelated variables. PCA is used for variable dimension reduction (feature extraction) and clustering purpose.

Sensor output profiles from the sensor array differ in response to targeted VOCs, therefore, pattern recognition techniques are used to analyze the data. The analysis involved two steps: signal preprocessing and pattern recognition. In the step of signal processing, the data is first normalized by the equation ΔR/Ri. After normalization, the baseline is corrected, and Principle Component Analysis is then employed for feature extraction and clustering purpose. Features extracted by PCA serve as the inputs to the neural network pattern recognizers. The PCA method also functions as a clustering technique in which the targeted VOCs are grouped into different clusters. In order to enhance the recognition rate, different neural network modules are applied to each cluster.

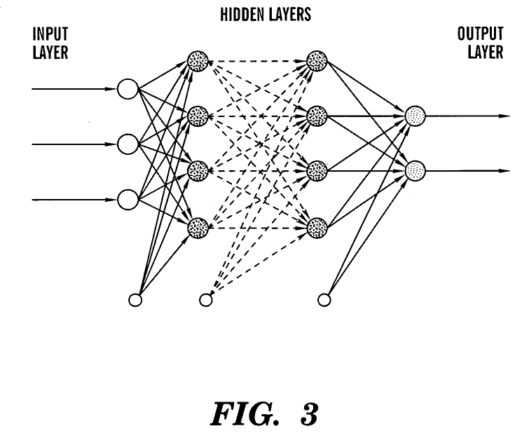

Multiple neural network modules with back propagation algorithm (BPN) are used for pattern recognition following the data processing. The BP algorithm is based on gradient descent in an error method which minimizes the mean square error between the network's output and the desired output for all input patterns. See Fausett, L., Fundamentals of Neural Networks, Architectures, Algorithms, and Applications, Prentice Hall, (1994), which is hereby incorporated by reference in its entirety. BPN is a multi-layer feed-forward network which has one input, one output, and at least one hidden layer. Each layer is fully connected to the succeeding layer, as shown, for example, in FIG. 3. During the learning process, the input vectors and the output of each neuron are computed layer by layer. The differences between the outputs of the final layer and the desired target vectors are back-propagated to the previous layer(s), modified by the derivative of the transfer function, and the connection weights are adjusted using the Widrow-Hoff learning rule. See Duda, R., et al., Pattern Classification, Wiley-Interscience, (2001), which is hereby incorporated by reference in its entirety.

Based on the cluster analysis result, an intelligent classifier with multi-module (or multi-level) neural network is constructed with each one dedicated to specific vapor group to perform vapor recognition. Each module consists of a Back Propagation Network (BPN) with its own suitable architecture. The advantage of the multi-module ANNs is to eliminate the need for accommodating all the identification knowledge for all target vapors in a single network. By using multiple networks, each network is trained for learning more specific knowledge on certain vapors. In this way, the overall correct recognition rate is enhanced by “multiple experts”.

A prototype sensor array device has been developed for the detection of acetone and other volatile organic vapors. A prototype PSA device is developed with battery-driven, non-invasive and cost-effective features capable of detecting, identifying, and quantifying acetone in human breath for diagnostics of diabetes. Breath testing is the least invasive of all diagnostic tests. Breath is a mixture of many different types of compounds, including VOCs, lipids, peptides, proteins and bacteria. The composition can differ between healthy and ill people. There are strong indications that many VOCs found in human breath may be markers of certain diseases. In the air breathed out of the lungs which contains approximately 15% O₂, 6% H₂O, and 5% CO₂, acetone is identified to be associated with diabetes. See Cheng, W., et al., J. Lab. Clin. Med., 133: 218 (1999); Ryabtsev, S., et al., Sensor and Actuators B, 59: 26 (1999); Zhang, et al., Q., Biosens Bioelectron., 15: 249 (2000); Koronczi, I., et al., IEEE Sensors Journal, 2: 254 (2002), which are hereby incorporated by reference in their entirety. In view of the complexity of composition in human breath, the sensors must be designed to be exceptionally fine-tunable in terms of nanoscale size, composition, surface, and spatial properties towards high sensitivity, low detection limit, high selectivity, and rapid response time.

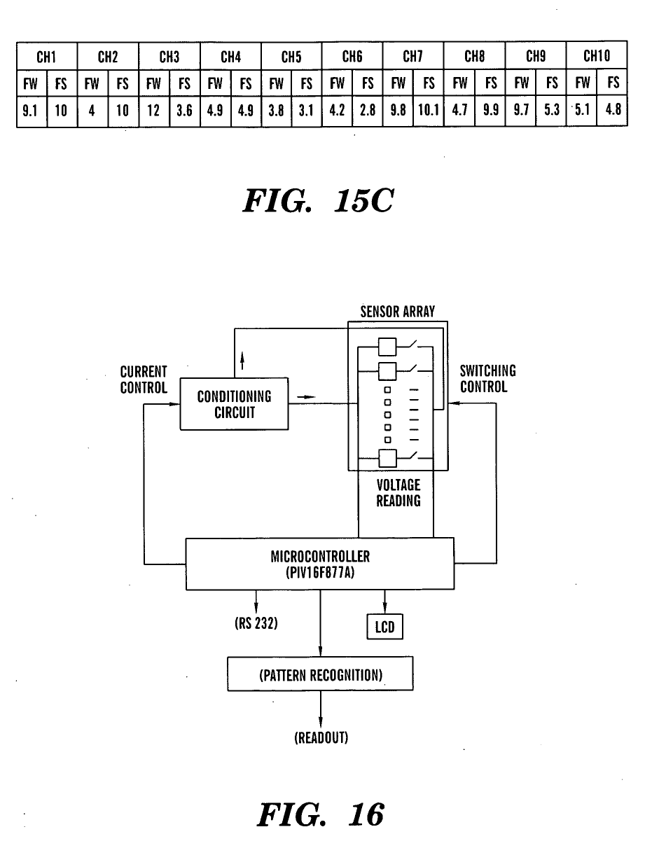

As illustrated in FIG. 16, the sensing array and pattern recognition device of the present invention may be packaged in a handheld device to detect the level of acetone in human breath accurately, rapidly, and without false alarms. The handheld device illustrated in FIG. 16 includes a data acquisition system, including a sensor array for detecting acetone. The sensor array may detect acetone in human breath or may detect acetone by sensing contact with a sensing platform. Similarly, sensing nanomaterials may be utilized and a sensor array trained to recognize patterns for detecting sample analytes indicative of acetone concentrations. Likewise, a combination of sensing methods and platforms may be used to provide redundant detection and backup methods of detecting acetone using the handheld device.

The handheld device also includes a conditioning circuit and a microcontroller. The microcontroller provides program instructions and controls for the sensor arrays and sensor platforms and processes detection readings from the sensor arrays and sensor platforms. The microcontroller also provides switching and current control over the sensor array and sensor platform and receives output data from the sensor array and sensor platform. The microcontroller further processes the output data from the sensor array and sensor platform and provides a readout or other indication to a user or to a data collection device. The handheld device may also include a storage device for retaining the output data and environmental factors during sampling, including detected levels of acetone, concentration ranges, analysis criteria, and other qualitative and quantitative evaluation criteria and performance factors.

Neural networks, cluster analysis, principal component analysis techniques, and other artificial intelligence systems may be coupled with the handheld device or otherwise implemented within the handheld device to further train the sensor array, sensor platform, and microcontroller and to provide a quantitative and qualitative indication of sampled acetone concentrations. Further, these artificial intelligence systems and networks may be used and trained to further refine sensor and platform selection criteria, sensor material selection criteria, microcontroller characteristics, and other component selection criteria based upon observed results.

The conditioning circuit may include filter networks and circuitry to modify the sensor array and sensor platform outputs to meet the operational requirements of the handheld device. The conditioning circuit may include noise reduction circuitry, phase equalization components, level stability circuits, frequency response correction circuitry, circuitry to correct impedance discontinuities, and other conditioning circuitry.

### EXAMPLES

### Example 1

### Chemicals

Hydrogen tetrachloroaurate trihydrate (HAuCl₄.3H₂O, 99%), silver nitrate (AgNO₃, 99+%), potassium bromide (KBr, 99+%), tetraoctylammonium bromide (TOA⁺Br⁻, 99%), decanethiol (DT, 96%), sodium borohydride (NaBH₄, 99%) were purchased from Aldrich. Alkyl dithiols (ADT, HS—(CH₂)ₙ—SH) included 1,3-propanedithiol (n=3, 99%), 1,5-pentanedithiol (n=5, 96%), 1,8-Octanedithiol (n=8, 97%), 1,9-nonadithiol (n=9, 95%), which were purchased from Aldrich and used as received. 1,10-decanedithiol (n=10, 90%) was purchased from TCI and used as received. Dicarboxylic acids (DCA, HO₂C—(CH₂)ₙ—CO₂H) included dodecanedioic acid (n=10, 99%) and 1,14-tetradecanedicarboxylic acid (n=14, 96%), which were purchased from Aldrich, and 1,12-dodecanedicarboxylic acid (n=12, 98%), 1,13-tridecanedicarboxylic acid (n=13, 97%), 1,16-hexadecanedicarboxylic acid (n=16, 97%), and 1,18-octadecanedicarboxylic acid (n=18, 99%), which were purchased from TCI and used as received. Solvents included hexane (99.9%) and toluene (99.8%) from Fisher, and ethanol (99.9%) from Aldrich. Water was purified with a Millipore Milli-Q water system. The tested organic vapors were generated from solvents of hexane (Hx, 99.9%, Fisher), benzene (Bz, 99.0%, Fisher), toluene (Tl, 99.9%, J. T. Baker).

### Example 2

### Synthesis of Nanoparticles

Au nanoparticles of 2 nm core size encapsulated with decanethiolate (DT) monolayer shells were synthesized by two-phase reduction of AuCl₄⁻ according to Brust's method and a synthetic modification. See Brust, M., et al., J. Chem. Soc., Chem. Commun., 7: 801 (1994) and Hostetler, M., et al., Langmuir, 14: 17 (1998), which are hereby incorporated by reference in their entirety. Details for the synthesis of gold nanoparticles (2.0±0.7 nm core size) were also previously described. See Maye, M., et al., Langmuir, 16: 490-497 (2000), which is hereby incorporated by reference in its entirety. AuAg alloy nanoparticles (3.0±0.5 nm core size) capped with DT monolayer shells were synthesized by a two-phase reduction of AuCl₄⁻ and AgBr₂⁻, details of which were recently reported. See Kariuki, N. N., et al., Langmuir. 20: 11240 (2004), which is hereby incorporated by reference in its entirety. AuAg nanoparticles with a Au:Ag ratio of 1:3 in the nanoparticle were synthesized and used in the present invention.

### Example 3

### Preparation of Thin Film Assembly

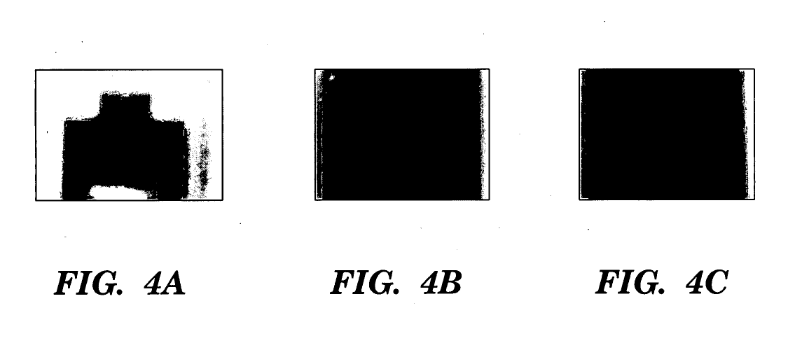

The general preparation of the thin films followed the one-step exchange-crosslinking-precipitation method reported for gold and AuAg nanoparticles. See Han, L., et al., Anal Chem., 73: 4441 (2001); Han, L., et al., J. Mater. Chem., 11: 1258 (2001); and Kariuki, N., et al., Chem. Mater., 18: 123 (2006), which are hereby incorporated by reference in their entirety. Briefly, the procedure involves immersion of substrates (e.g., glass, electrodes etc.) into a hexane solution of DT-capped Au (30 μM) and ADT (50 mM) for the ADT-Au assembly, or a mixture of hexane solution of DT-capped AuAg nanoparticles (1.0 μM) and ethanol or tetrahydrofuran solution of DCA (20 mM) for the DCA-AuAg assembly. The reaction was carried out at room temperature. ADT or DCA function as a mediator or cross-linking agent. The mediator to nanoparticle ratio was controlled, typically about 100:1. The pre-cleaned substrates or devices were immersed vertically into the assembly solution to ensure that the film formed was free of powder deposition. At a controlled immersion time, the film-deposited substrates were immersed and immediately rinsed thoroughly with hexane and dried under nitrogen before the characterization. The chain length for the thin films is denoted according to the number of —CH₂— units (n) in ADT, which include n=3, 5, 8, 9, and 10, or in DCA, which include n=10, 12, 13, 14, 16, and 18. FIG. 4A-C shows photos for both ADT-Au and DCA-AuAg thin films formed on IME devices. FIG. 4A shows an interdigitated microelectrode (IME). FIG. 4B shows ADT-Au thin film on IME (n=10). FIG. 4C shows DCA-AuAg thin film on IME (n=16). The films were uniform and the thickness could be controlled.

### Example 4

### Devices and Measurements

Sensor response measurements were performed using an array of IME devices, with 100 pairs of gold electrodes of 200 μm length, 10 μm width and 5 μm spacing on a 1-mm thick glass substrate (thickness of the Au electrodes: 100 nm). Details for the microfrabrication of the IMEs were reported previously. See Wang, L., et al., Sensors., 6: 667 (2006), which is hereby incorporated by reference in its entirety. The thickness of the coating was below or close to the finger thickness. A computer-interfaced multi-channel multimeter (Keithley, Model 2700) was used to measure the lateral resistance of the nanostructured coating on IME. All experiments were performed at room temperature, 22±1° C. N₂ gas (99.99%, Airgas) was used as reference gas and as diluent to change vapor concentration by controlling mixing ratio. The gas flow was controlled by a calibrated Aalborg mass-flow controller (AFC-2600). The flow rates of the vapor stream were varied between 3 and 99 mL/min, with N₂ added to a total of 100 mL/min. The vapor generating system consisted of a stainless steel multi-channel linked to different vapor bubblers (Teflon material). The design of the multi channel module was such that the dead-volume was kept to a minimum negligible value. The sensor array system with modular platform components has allowed testing vapor responses of different nanostructured array elements with minimum dead-volume and virtually no cross-contamination. The vapor stream was produced by bubbling dry N₂ gas through a selected bubbler (valve controlled) of the vapor solvent using the controller to manipulate vapor concentration.

The IME devices were housed in a Teflon chamber with tubing connections to vapor and N₂ sources; the electrode leads were connected to the multimeter. Nitrogen was used as carrier gas. Different concentrations of vapors were generated using an impinger system. At the beginning of the experiment, the test chamber was purged with pure nitrogen for a 1 hour to ensure the absence of air and also to establish the baseline. The test chamber was purged with N₂ and the analyte vapor alternately. A series of vapor concentration was tested. The vapor concentration in the unit of ppm moles per liter was calculated from the partial vapor pressure and the mixing ratio of vapor and N₂ flows. Details of the measurement protocols were described previously. See Han, L., et al., Anal Chem., 73: 4441 (2001); Han, L., et al., Sens. Actuators B., 106: 431 (2005); Shi, X., et al., Sens. Actuators B., 117: 65 (2006); and Wang, L., et al., Sensors., 6: 667 (2006), which are hereby incorporated by reference in their entirety. ΔR is the difference of the maximum and minimum values of the resistance in response to vapor exposure, and Rᵢ is the initial resistance of the film. See Severin, E. J., et al., Anal. Chem., 72: 2008 (2000), which is hereby incorporated by reference in its entirety. The sensitivity data were based on the relative differential resistance change, ΔR/Rᵢ, versus vapor concentration, C (ppm). The concentration is given in ppm (M), which can be converted to ppm (V) by multiplying a factor of 24.5. See Han, L., et al., Anal. Chem., 73: 4441 (2001); Han, L., et al., Sens. Actuators B., 106: 431 (2005); Shi, X., et al., Sens. Actuators B., 117: 65 (2006); and Wang, L Y, et al., Sensors., 6: 667 (2006), which are hereby incorporated by reference in their entirety.

The structural and morphological properties of most of the ADT-Au and the DCA-AuAg thin films have been characterized by TEM and FTIR techniques in previous reports. See Leibowitz, F., et al., Anal Chem., 71: 5076 (1999); Han, L., et al., J. Mater. Chem., 11: 1258 (2001); and Kariuki, N., et al., Chem. Mater., 18: 123 (2006), which are hereby incorporated by reference in their entirety.

### Example 5

### Response Characteristics of ADT-Au Array

The electrical conductivity of several nanoparticle thin film assemblies on an IME was shown earlier work to be dependent on particle size and interparticle properties. See Han, L., et al., Anal. Chem., 73: 4441 (2001) and Han, L., et al., Chem. Mater., 15: 29 (2003), which are hereby incorporated by reference in their entirety. The measured resistance (R_{Ω}) is related to the lateral conductivity (σ) of the film by the relationship of σ=(1/R_{Ω})(w/dL), where w is the gap width of the array electrodes, L is the length of the electrodes, and d is the film thickness. The resistance and the thickness of the film can be controlled by assembly time and chain length. The initial resistance (R_{Ω}) was found to decrease exponentially with assembly time for ADT-Au films, reflecting the increase of film thickness.

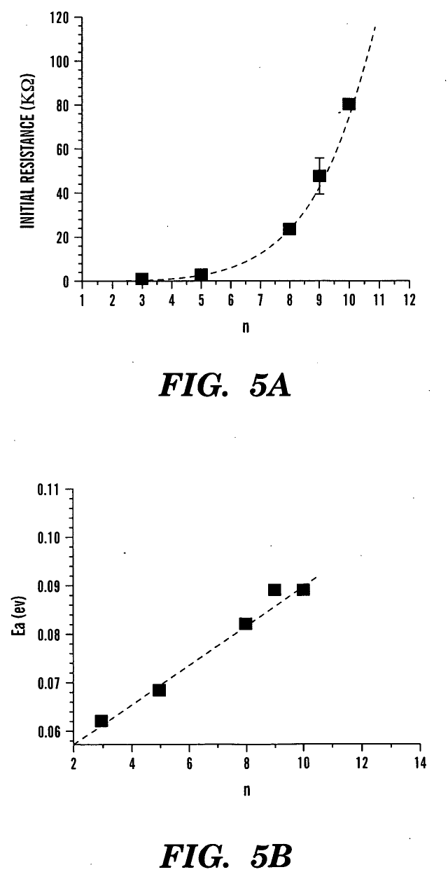

FIGS. 5A-B show a representative set of initial resistance data measured for thin film assemblies of DT-capped gold nanoparticles mediated by ADTs of different chain lengths. The resistance clearly displays an exponential rise vs. the chain length. This relationship is quite consistent with the overall electronic conduction mechanism in which the electron hopping and/or electron tunneling are dependent on the interparticle distance. The electrical conductivity depends on the core radius (r), interparticle distance (d), and dielectric constant of interparticle medium (ε) by a thermally-activated conduction path

$$σ = {σ}_{0} exp ( - \frac{{E}_{a}}{RT} ) \qquad ( 1 )$$

where the activation energy (Eₐ) is

$${E}_{a} = 0.5 {e}^{2} \frac{{r}^{- 1} - {( r + d )}^{- 1}}{4 {πɛɛ}_{0}} \qquad ( 2 )$$

See Abeles, B., et al., Adv. Phys., 24: 407 (1975); Bethell, D., et al., J. Electroanal. Chem., 409: 137 (1996); and Brust, M., et al., Langmuir, 14: 5425 (1998), which are hereby incorporated by reference in their entirety.

Each addition of —CH₂— in the alkyl chain leads to an increase of 0.13 nm spacing. The interparticle distance (d) is related to chain length (n) by the relationship d=1.5+0.13 n (nm). The remarkable fitting by eqn. 1-2 (R_{Ω}∝1/σ), as shown in FIG. 5A, demonstrates that the electrical conduction in the thin film assembly follows a thermally-activated conduction path. Based on measurement of the temperature dependence of the conductivity, a further comparison of the activation energy for the thin films derived from different chain lengths shows an approximate linear relationship, yield 0.004 eV per methylene unit. See FIG. 5B. The observation of the linear relationship is in agreement with those for layer-by-layer stepwise assemblies of gold nanoparticles reported by Brust and co-workers. See Bethell, D., et al., J. Electroanal. Chem., 409: 137 (1996) and Brust, M., et al., Langmuir, 14: 5425 (1998), which are hereby incorporated by reference in their entirety.

Detailed assessment of the chainlength dependence of the electrical conductivity demonstrates that the electrical properties can be fine tuned by the interparticle distance through the mediator linkers, which constitute the basis for the design of the sensor array films of the present invention, as detailed below.

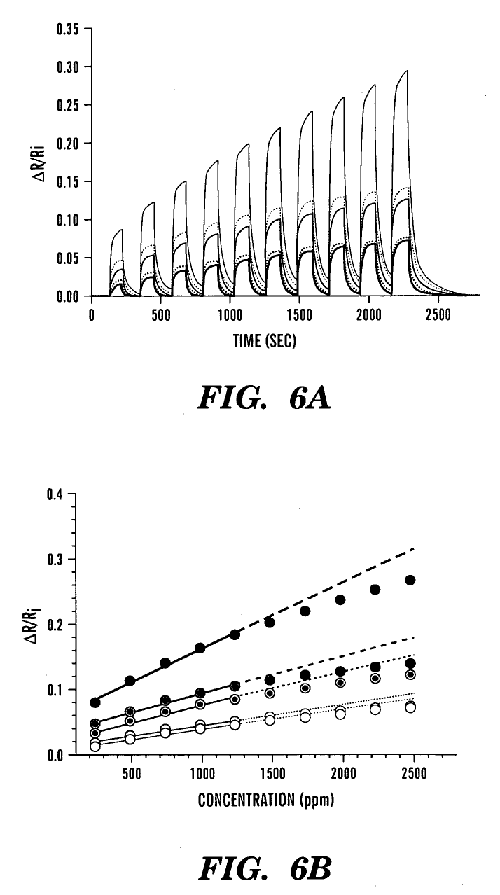

An example array consisting ADT-Au thin films with n=3, 5, 8, 9, and 10 was examined. FIGS. 6A-B show a typical set of sensor response profiles for this sensor array, along with the dependence of the sensor response on vapor concentration. The response profile features an increase in ΔR/Rᵢ upon exposure to the vapor which returns to baseline upon purge with nitrogen. The response is rapid and reversible. In most cases, the responses increased linearly with vapor concentration when the concentration is not too high. The slope serves as a measure of the response sensitivity. Deviation from the linear relationship occurs when the vapor concentration is high, which is due to the existence of a saturation effect and or the complication of both bulk and surface adsorption phenomena. See Han, L., et al., Anal Chem., 73: 4441 (2001), which is hereby incorporated by reference in its entirety. For the convenience of an overall assessment, the linear approximation for assessing the sorption data was used.

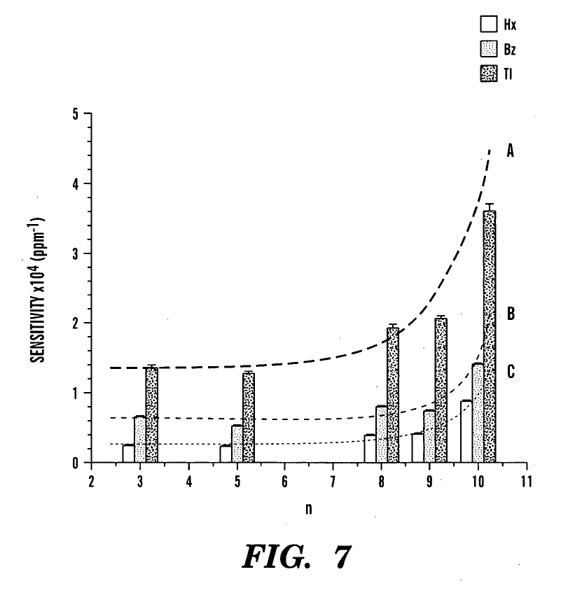

FIG. 7 compares the vapor response sensitivities of the nanoparticle thin film assemblies derived from different chain lengths. The data reveal a general trend of an exponential rising for the response sensitivity vs. chain length, demonstrating the viability of fine tuning of the response sensitivity of the thin film coated chemiresistor sensors by interparticle distance in the nanostructure. This dependence is less significant for thin films derived from short chain mediators.

The issue on the effect of film thickness on the sensor response sensitivity was also addressed by examining the dependence of the sensitivity vs. the relative thickness. The sensitivity was found to be dependent on the thickness only for very thin films with a relative thickness <100. For relatively thicker films, the sensitivity is essentially independent on the film thickness. The fact that the films tested were all relatively thicker substantiated the comparison of the response sensitivity data in FIG. 7.

### Example 6

### Response Characteristics of DCA-AuAg Array

The sensing array consisting of DCA-mediated thin film assemblies of DT-capped AuAg nanoparticles provided another system for the investigation of the effect of chain length on the sensor response sensitivity. An example array consisting of DCA-AuAg thin films with n=10, 12, 13, 14, 16, and 18 was examined. These films differ from each other in terms of the chain length of the mediator molecule, and therefore the interparticle spacing. The initial resistance values for the IME array of thin films were also measured. Similar to that for the array of ADT-Au thin films, the resistance of the thin films was found to decrease with assembly time, i.e., film thickness. The difference in the initial resistances reflects the difference in film thickness. The dependence of the resistance on the chain length of DCAs was found to display an exponential rise vs. the chain length, similar to the case of ADT linked thin films of Au nanoparticles.

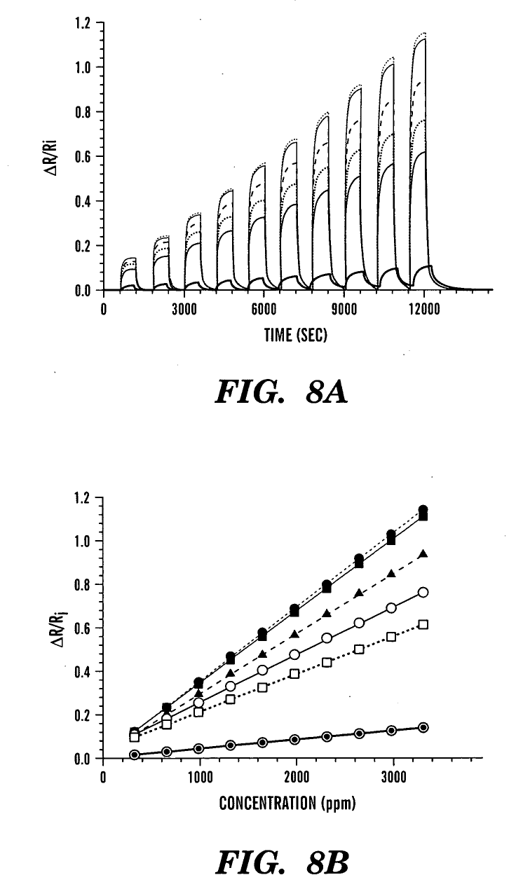

The response profiles for a sensing array of six DCA-AuAg thin film materials on IME devices, i.e., n=10, 12, 13, 14, 16, and 18, in response to a series of volatile organic vapor analytes were first examined. FIGS. 8A-B show a representative set of response characteristics for a 6-sensor array. The response profiles for a selected set of vapor concentrations are displayed (see FIG. 8A), and the corresponding response sensitivities are plotted against concentration (see FIG. 8B). Again, the sensing array displays linear responses to concentrations of the vapors. While the response profiles of the same vapors at different films are similar, the response sensitivities vary dramatically, as evidenced by the differences in the slopes of the linear relationships. In contrast to those observed for ADT-Au films, little deviation from the linear relationship was observed, suggestive of the lack of a saturation effect and or the complication of both bulk and surface adsorption phenomena for this type of thin films.

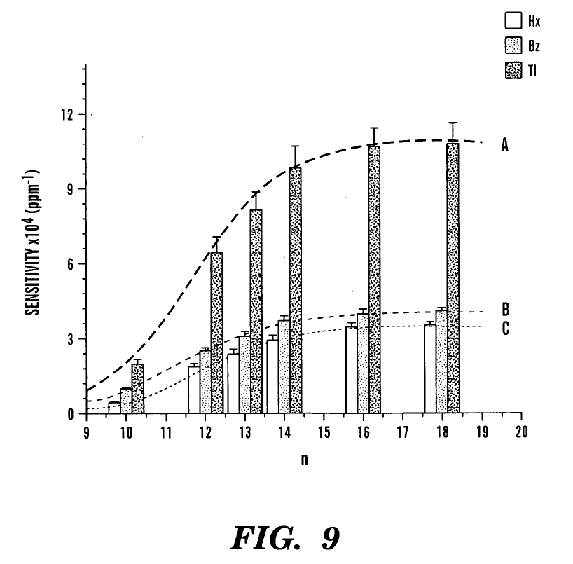

FIG. 9 shows the dependence of the sensor response sensitivities on chain length. The sensitivities to several typical VOCs are found to exhibit an exponential rise to a maximum as a function of the chain length, demonstrating the sensitivity of the sensor response to interparticle distance in the nanostructure. The observed dependence reflects in part an increase of the interparticle nanoscale porosity with chain length, and in part a decrease of the electronic conductivity of the thin film assembly with chain length. The chain length dependence of the electronic conductivity was in fact demonstrated in a previous report. See Leibowitz, F., et al., Anal Chem., 71: 5076 (1999); Han, L., et al., J. Mater. Chem., 11: 1258 (2001); Kariuki, N., et al., Chem. Mater., 18: 123 (2006); Bethell, D., et al., J. Electroanal. Chem., 409: 137 (1996); and Brust, M., et al., Langmuir, 14: 5425 (1998), which are hereby incorporated by reference in their entirety. This finding is significant because it suggests the nanostructured sensing properties can be fine tuned at the molecular level. Further insights into the sensor response and chain length correlation are gained by thermodynamic analysis of the sensor response characteristics and statistical analysis of the sensor array performance.

### Example 7

### Thermodynamic Assessment of Chain Length Dependence of Response Characteristics

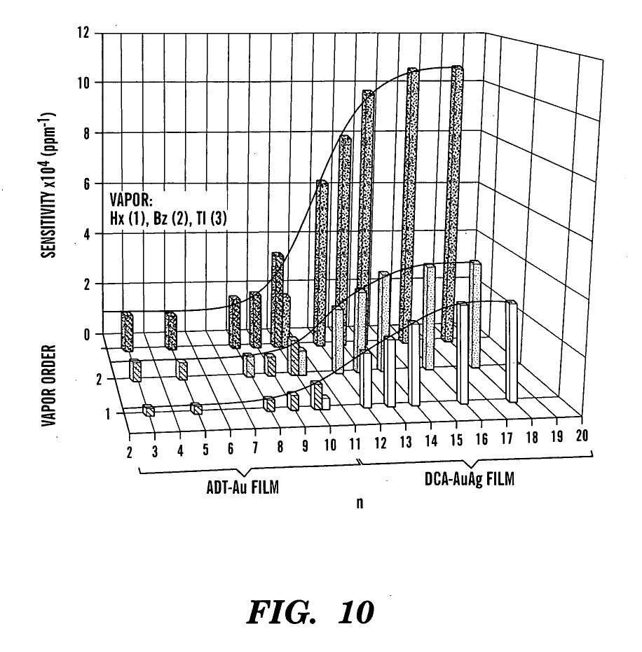

Since the difference in particle sizes between Au and AuAg nanoparticles is small, these two types of particles can be approximately considered as nano-building blocks of similar sizes whereas the combination of the variable number of methylene units in the linking alkyl chain (ADT and DCA) and the fixed number of methylene units in the capping alkyl chain provide a defined tunability in interparticle spacing. FIG. 10 shows response data obtained by combining the ADT-Au and DCA-AuAg thin films with different chain length. The observed general trend is characteristic of sigmoidal feature rising to a maximum, demonstrating that the interparticle spatial properties played a dominant role in the sensor response characteristics.

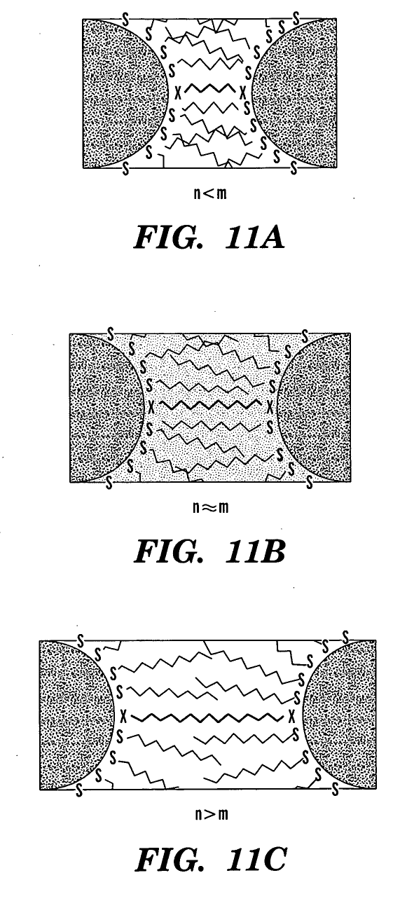

The fact that those films with longer alkyl chains display higher response sensitivity than those with shorter alkyl chains can be explained by the larger volume fraction of organic structures in the long chain case which favor the sorption of the organic vapor into the film. The partition of vapor molecules in the film leads to an increased interparticle spacing, which changes the conductivity more significantly for the longer chain films than that for the shorter chain films. To understand the sigmoidal feature for the dependence of the sensitivity on the chain length where the most significant change was observed to occur in the range of n=9˜13, the thermodynamic equilibrium for vapor sorption in the films of different chain lengths was further considered. Based on the vapor partition equilibrium constant (Kₙ), Kₙ=Cₙ(film)/Cᵥ(vapor), where Cₙ is the vapor concentration in the film at the vapor phase concentration (Cᵥ, Cᵥ=ΔR/(Rᵢ×S (response sensitivity)), the relative concentration ratio, in consideration of the free energy of adsorption, ΔG_{ads}=−RT ln K, yields, Cₙ/C_{n′}=Kₙ/K_{n′}=exp(−Δ(ΔG)/RT). By a rough estimate based on the difference of cohesive energy between two neighboring chain length (e.g., n=10 and n′=9), i.e., Δ(ΔG)=˜0.8 kcal/mol, the C₁₀/C₉ ratio would be 4. See Nuzzo, R. G., et al., J. Am. Chem. Soc., 112: 558 (1990), which is hereby incorporated by reference in its entirety. The increase of 1 methylene unit would lead to ˜4× more vapor sorption into the film, which is in fact quite consistent with the relative change of the thin film resistance in response to exposure of the vapor tested (see FIGS. 6 and 7). The relative change in cohesive energy in the n=9˜13 region may inherently be linked to the intriguing observation in FIG. 10 that the significant dependence occurs in the range of n=9˜13, beyond which the dependence becomes less significant. This finding reflects the interparticle spatial or structural effect on the relative change of the electrical conductivity due to the relative length differences of the interparticle —(CH₂)ₙ— structures defined by both the mediating and the capping (or templating) molecules, leading to subtle differences in thermodynamic driving forces for the vapor-nanostructure interactions. See FIGS. 11A-C. For n=9˜13, the molecular length falls in the vicinity of m=9, the vapor molecules enter a well-interdigitated mediating/capping alkyl structures. The perturbation of the interparticle distance is thus very sensitive to the mediator chain length. When n<m, the electrical conductivity of the film is relatively high and the organic volume fraction is relatively small so that the alkyl chains are not well interdigitated. In this case, the change in conductivity in response to the sorption of vapor molecules into the film is less sensitive to n in comparison with those for n≈m. When n>m, the conductivity is relatively low and the organic volume fraction is relatively large so that the alkyl chains can not be well interdigitated. In this case, the change in conductivity in response to the sorption of vapor molecules into the film is also less sensitive to n in comparison with those for n≈m.

To gain further insights into the thermodynamic factor dictating the sorption equilibrium, the response kinetics are analyzed by considering the sorption of vapor (e.g., hexane):

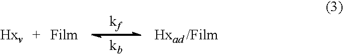

where hexane in the vapor phase, Hxᵥ, adsorbs at a “binding site” in the film forming Hx_{ad}/Film. k_{f} and k_{b} define the forward and backward adsorption rate constants, respectively. By assuming a Langmuir adsorption isotherm, which is reasonable for processes involving only hydrophobic interactions, the surface coverage (θ) (θ=Γₜ/Γ₀, Γₜ and Γ₀ represent coverage at time t and maximum coverage) at a given vapor concentration (Cᵥ) can be derived as

θ=a[1−exp(−bt)] (4)

where a=Cᵥ/(Cᵥ+K⁻¹), K=k_{f}/k_{b}, b=k_{f}Cᵥ+k_{b}. On the basis of the response data characteristics, it is reasonable to relate the q(t) to the measured change in resistance, dR/dt=Fθ(t), where F is a proportionality factor. By fitting the transient response data for different vapor concentrations by equation 4, values of F and the rate constants (k_{f} and k_{b}) can be determined for each film, which allow the equilibrium constant K and ΔG_{ads} to be estimated. See Table 1 below.

TABLE 1
Table 1. Results based on curve fittings of the data.

| Chain length (n) | F | k₊ | k₋ | K | ΔG |
|---|---|---|---|---|---|
| 5 | 0.111 | 50.0 | 0.069 | 721 | 3.88 |
| 8 | 0.167 | 68.8 | 0.080 | 857 | 3.98 |
| 9 | 0.188 | 125.0 | 0.134 | 930 | 4.03 |
| 12 | 0.838 | 13.1 | 0.051 | 258 | 3.28 |
| 14 | 1.518 | 11.5 | 0.057 | 200 | 3.13 |
| 16 | 1.251 | 15.7 | 0.051 | 317 | 3.40 |

The ΔG_{ads} values, −4.0-3.4 kcal/mol, were found to fall in between those expected for the condensation energy of hydrocarbons (6 kcal/mol) and those reported for the cohesive energy of alkyl chains (i.e., 1.4˜1.8 kcal/mol). See Nuzzo, R. G., et al., J. Am. Chem. Soc., 112: 558 (1990), which is hereby incorporated by reference in its entirety. This result is consistent with the nature of the hydrophobic interaction of hexane vapor with the alkyl network in the nanoparticle thin film assembly. Interestingly, the values of Kₙ for shorter alkyl chains are found to be larger than those of K_{n′} for longer alkyl chains. The ΔG_{ads} values display a subtle transition at n=˜10 from ˜−4.0 kcal/mol for shorter chains to ˜−3.4 kcal/mol for longer chains. This transition coincides with the transition of the response sensitivity (see FIG. 10), reflecting the important role played by the thermodynamic factors in the nanostructured sensing properties.

These findings have significant implications to the design of sensing nanostructures in terms of interparticle spatial and electrical properties. It is apparent the combination of mediator (—(CH₂)ₙ—) and capping (—(CH₂)ₘ—) molecules in the nanostructured thin films dictate particle size that determines the thermodynamic equilibrium for the vapor sorption. The study of the effect of the variation of the capping molecule X—(CH₂)ₘCH₃ on the sensing properties is expected to provide important insights into the fine engineering of the interparticle mediating/templating interactions of the nanostructured sensing materials.

### Example 8

### Statistical Assessment of the Chain Length in Sensor Array Performance

To further assess the correlation between interparticle spacing and the sensor response characteristics, Analysis of Variance (ANOVA) techniques were used to analyze the data in terms of sensitivity and selectivity. A general full factorial experiment was conducted to assess the sensitivity and selectivity, which involved two factors: the interparticle spatial parameter and the vapor type. The selectivity characteristics were evaluated by calculating the Euclidean distance among vapor response curves. Finally, the thin films with different chain length were investigated with Principal Component Analysis (PCA) method. The following sections discuss the analysis results in terms of the sensitivity, selectivity, and PCA results.

The general full factorial experiments for ADT-Au and DCA-AuAg sensor arrays were designed with the experiment parameters summarized in Table 2, below, in which each experiment has two factors: interparticle spatial parameter with 5 levels for ADT-Au and 6 levels for DCA-AuAg sensors, and vapor with 3 levels for each type of sensors. The normalized response sensitivities of the ADT-Au films (five different chain length designs) and the DCA-AuAg films (six different chain length designs) to the three different vapors (hexane, benzene, and toluene) were used as the performance measures for the evaluation. Three duplicate measurements on the responses for each of ADT-Au and DCA-AuAg films were taken. The average of the three duplicate measurements serves as the experimental response.

TABLE 2
Table 2. General full factorial design
for ADT-Au and DCA-AuAg sensors.

| | (I) ADT-Au film array | | | | | (II) DCA-AuAg film array | | | | | |
|---|---|---|---|---|---|---|---|---|---|---|---|
| Level | 1 | 2 | 3 | 4 | 5 | 1 | 2 | 3 | 4 | 5 | 6 |
| Factor I: Chain | 3 | 5 | 8 | 9 | 10 | 10 | 12 | 13 | 14 | 16 | 18 |
| length | | | | | | | | | | | |
| Factor II: Vapor | Hx | Bz | Tl | | | Hx | Bz | Tl | | | |

The experimental results were analyzed with ANOVA method. The factors (or parameters) with P-value smaller than a significant level a were considered as significant factors. Table 3 summarizes the ANOVA results of sensitivity for the two different films respectively. The P-values for interparticle spatial parameter and vapors were all found to be smaller than the significant level (α=0.05). It is therefore concluded that both the interparticle spatial parameter and the vapor type have significant influence on the sensitivity of the thin films.

TABLE 3
Table 3. ANOVA results for the sensitivity
of ADT-Au and DCA-AuAg sensing films.

| | | | | | | |
|---|---|---|---|---|---|---|
| Source | Degrees | Sequential | Adjusted | Adjusted | | |
| of | of | Sum of | Sum of | Mean | | P- |
| Variation | Freedom | Squares | Squares | Square | F₀ | value |

ADT-Au

| | | | | | | |
|---|---|---|---|---|---|---|
| n | 4 | 235.25 | 235.25 | 58.81 | 5.19 | 0.023 |
| Vapor | 2 | 606.21 | 606.21 | 303.10 | 26.75 | 0 |
| Error | 8 | 90.65 | 90.65 | 11.33 | — | — |
| Total | 14 | 932.11 | — | — | — | — |

DCA-AuAg

| | | | | | | |
|---|---|---|---|---|---|---|
| n | 5 | 6291.2 | 6291.2 | 1791.5 | 61.67 | 0.007 |
| Vapor | 2 | 13215.8 | 13215.8 | 6607.9 | 32.81 | 0 |
| Error | 10 | 2013.9 | 2013.9 | 201.4 | — | — |
| Total | 17 | 21521.0 | — | — | — | — |

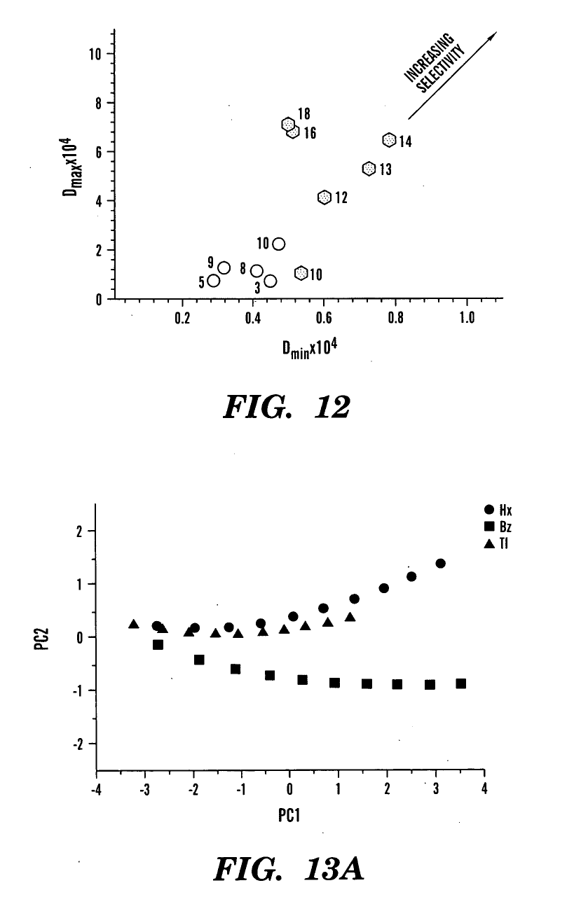

To understand the correlation between the interparticle distances and the sensor properties, the above experimental data were further analyzed using a selectivity evaluation technique. See Han, L., et al., Sens. Actuators B., 106: 431 (2005) and Shi, X., et al., Sens. Actuators B., 117: 65 (2006), which are hereby incorporated by reference in their entirety. The selectivity characteristic of a thin film is measured with the Euclidean distances among the response curves for different vapors. The minimum distance (Dₘᵢₙ) describes how well the two closest vapors response curves can be distinguished by a film, whereas the maximum separation distance (Dₘₐₓ) characterizes the film's highest separation capability. The selectivity characteristics, Dₘᵢₙ and Dₘₐₓ are calculated and summarized in FIG. 12. A larger value for the measures means that the film has better capability to distinguish different vapors. A compromised balance between Dₘᵢₙ and Dₘₐₓ would suggest that an array consisting of thin films with n=14, 13, 12, 18, and 16 is desired.

It is observed that the selectivity of the thin film array is also influenced by chain length. To further investigate the effect of interparticle spatial parameter on the film's selectivity characteristics, the experimental results of Dₘₐₓ and Dₘᵢₙ were analyzed with ANOVA method. Tables 4 and 5 and summarize the ANOVA results of Dₘₐₓ and Dₘᵢₙ for the two different types of films. In the ANOVA tables, p-values are all smaller than significant level (0.05) for both Dₘₐₓ and Dₘᵢₙ, which indicates that the chain length significantly influences the separation capability. The results suggest that the separation capability of the thin film array could be potentially enhanced by specifying the chain length for both ADT-Au and DCA-AuAg sensor arrays.

TABLE 4
Table 4. ANOVA results for the Dₘₐₓ measure
of ADT-Au and DCA-AuAg films.

| | | | | | |
|---|---|---|---|---|---|
| Source of | Sum of | Degrees of | Mean | | |
| Variation | Squares | Freedom | Square | F₀ | P-value |

ADT-Au

| | | | | | |
|---|---|---|---|---|---|
| n | 4 | 1177.540 | 294.385 | 565.680 | 0.000 |
| Error | 31 | 16.133 | 0.520 | — | — |
| Total | 35 | 1193.673 | — | — | — |

DCA-AuAg

| | | | | | |
|---|---|---|---|---|---|
| n | 5 | 4155.400 | 831.100 | 17.140 | 0.000 |
| Error | 10 | 484.900 | 48.500 | — | — |
| Total | 15 | 4640.400 | — | — | — |

TABLE 5
Table 5. ANOVA results for the Dₘᵢₙ
measure ADT-Au and DCA-AuAg films.

| | | | | | |
|---|---|---|---|---|---|
| Source of | Degrees of | Sum of | Mean | | |
| Variation | Freedom | Squares | Square | F₀ | P-value |

ADT-Au

| | | | | | |
|---|---|---|---|---|---|
| n | 4 | 5.236 | 1.309 | 7.880 | 0.035 |
| Error | 4 | 0.665 | 0.166 | | |
| Total | 8 | 5.901 | | | |

DCA-AuAg

| | | | | | |
|---|---|---|---|---|---|
| n | 5 | 10.346 | 2.069 | 8.020 | 0.020 |
| Error | 5 | 1.290 | 0.258 | | |
| Total | 10 | 11.636 | | | |

Principal Component Analysis (PCA) was employed to evaluate the performance of the test sensor array with films of different chain lengths. The purpose of the PCA analysis is to visualize the capability of a sensor array in distinguishing different vapors. PCA is a mathematical method that converts a large number of potentially correlated variables into relatively small number of uncorrelated variables that can serve as the features for distinguishing different vapors. PCA is used in this work to reduce variable dimensions (feature extraction) and visualize the classification result of the test vapors.

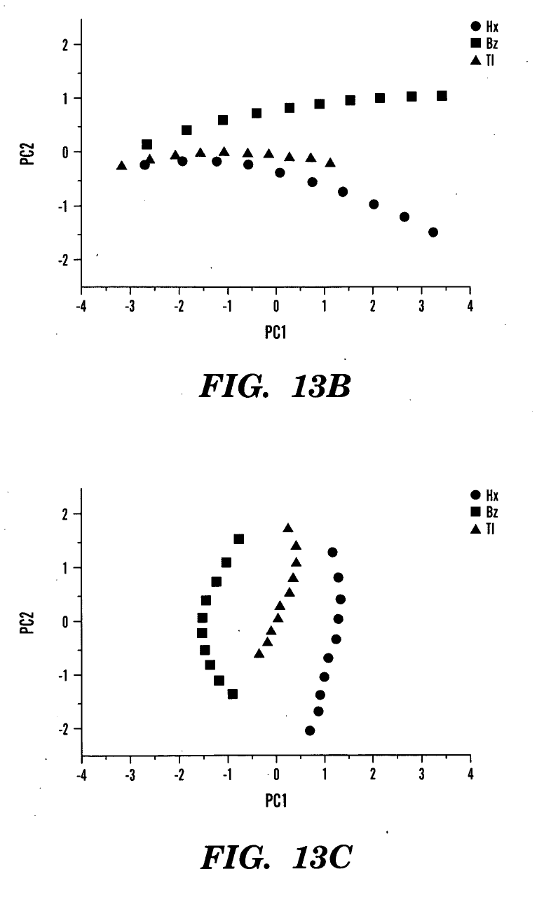

To establish the relation between the classification capability of the sensor array and the interparticle spatial parameter of thin film, sensor arrays with different combinations (e.g., 2 films, 3 films, 4 films, etc) of the thin films with variant chain length for different vapors have been tested. The results showed that the classification capability of each array is highly dependent on the specific combination. To illustrate this assessment, three sensor arrays from the following combinations were chosen. Array A consists of ADT-Au and DCA-AuAg films with shorter chain length (ADT-Au (n=3 and 5) and DCA-AuAg (n=12 and 13); Array B consists of ADT-Au films with shorter chain length and DCA-AuAg films with longer chain length (ADT-Au (n=3 and 5) and DCA-AuAg (n=16 and 18)). Array C consists of ADT-Au and DCA-AuAg films with longer chain length (ADT-Au (n=9 and 10) and DCA-AuAg (n=16 and 18)). The response data of the three arrays to three test vapors (hexane, benzene, and toluene) were analyzed. The first two components of each array were utilized to identify the different vapor patterns. FIGS. 13A-C show the PCA score plots for each of the three vapors in the PC₁-PC₂ plane, which was obtained by performing PCA analysis on the normalized responses of the sensor arrays at 10 different concentration levels. The three different vapor response patterns can be well separated from each other with array C (see FIG. 13C). For arrays A (FIG. 13A) and B (FIG. 13B), while the response pattern for Bz is well separated from those for Hx and Tl, the response patterns for Hx and Tl are overlapped at lower concentration region. One of the implications of this observation is that the interparticle spatial parameter of the thin films affects the classification capability of the sensor array. By appropriately selecting the combination of interparticle spatial parameter for the thin film in the array system, the classification capability is significantly enhanced.

### Example 9

### Detection of Acetone

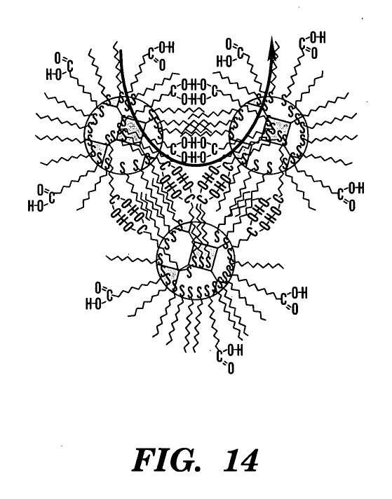

The design and assembly of array sensing nanomaterials are based on a rational combination of nanoparticle size, composition, interparticle distance, interparticle physical/chemical property, and overall film thickness. The organic shell molecules, linkers, mediators and surfactants are functionalized using different functional groups, including, —CH₃, —OH, —CO₂H, —NH₂, etc. FIG. 14 shows an illustration of film assembly of nanoparticles (MUA-Auₙₘ) as sensing materials.

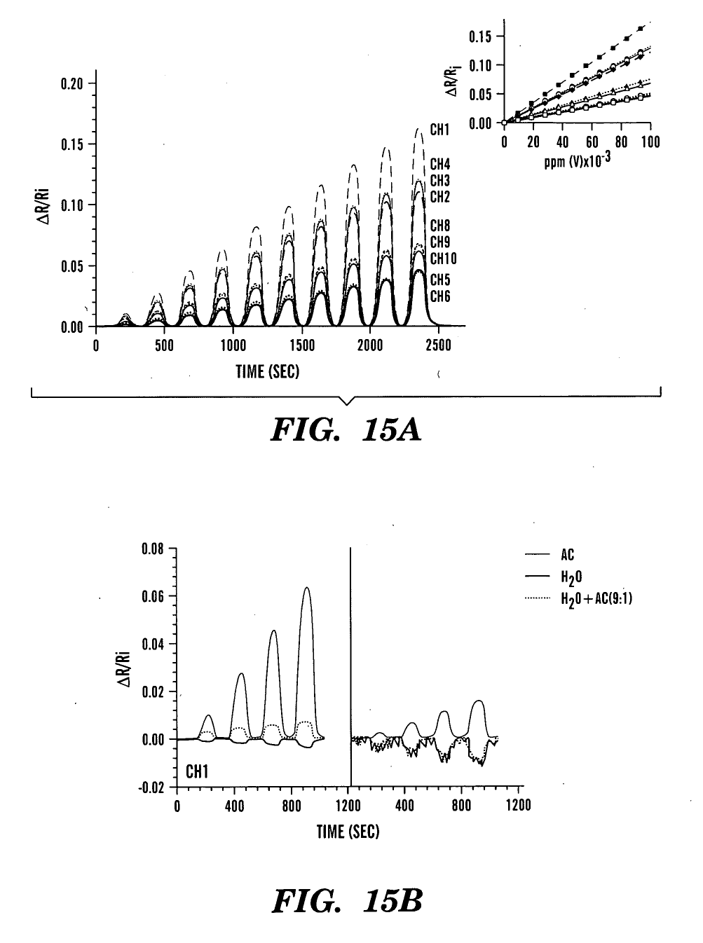

FIGS. 15A-B shows a set of data for the detection of acetone of various concentrations on a 10-channel IME-array of different design parameters which are coated with MUA-mediated thin film assemblies of gold nanoparticles. It exhibits linear response vs. acetone concentration (see FIG. 15A). Most importantly, the initial results have demonstrated the viability of achieving clear selectivity between acetone and water (see FIG. 15B), which a major difficulty in many other sensor technologies. The detection limit reached 1 ppm to 10 ppb depending on the actual combination of the IME design parameters and the array nanomaterials structures. The sensor responses to mixtures of CO₂, H₂O, and ketones are established in combination with pattern recognition in terms of identification and quantification.

The fabrication of a handheld prototype device integrates sensing arrays, pattern recognition and electronic readout components, producing a compact array of IMEs on chips. The response profiles or patterns of a sensor array to a certain set of VOCs are utilized for identification of the vapors. The concentration of different vapors can be estimated by algorithm using the responses. Codes for pattern recognition and concentration estimation algorithm are developed and integrated into the device microprocessor. Artificial neural networks (ANNs) for pattern recognition are used in processing the sensing array data. ANNs are massively parallel computing systems mimicking human neurobiological information-processing activities.

The selected sensor array is integrated with hardware, software, signal processing, and ANN pattern recognition and concentration algorithm to perform the detection and quantification task with optimized overall performance such as sensitivity, selectivity, miniature, and power consumption. In the hardware design, the multiple electrical signals from IME sensor array device are collected by a data acquisition system, which is controlled by a microcontroller for data storage and display. See FIG. 16. This hardware design extends its resistance measurement ranges so that it can allow wide applications of sensing materials. To automatically perform the functions of data processing, pattern recognition, and concentration quantification, a FPGA or DSP microprocessor are also integrated into the circuit board with customized programs. The concentration of acetone is correlated to the level of glucose. An alarm system is triggered when the glucose level exceeded the limits. The historical data can be stored and statistics (mean, variations, and trend) can be displayed.

### Example 10

### Pattern Recognition of Sensor Array Data

On the basis of the preliminary results, it is important to select the optimum combination of the sensor array from the different array candidates in order to achieve the best selectivity. Linear Discriminant Analysis (LDA) is used to facilitate the optimum selection of sensor elements from our initial candidates by investigating how films in each group (MUA-Au₇₋ₙₘ, MUA-Au₂₋ₙₘ, and NDT-Au₂₋ₙₘ) contribute to vapor separation. The films are selected based on their separation distance between water and mixture of water and acetone. Out of initial 10 candidates, the 5 best sensors selected and their DA distances are shown in Table 6.

TABLE 6
Table 6. Linear Discriminant Analysis (LDA) distance
between water and mixture for the selected films.

| MUA-Au₇₋ₙₘ #2 | MUA-Au₂₋ₙₘ #1 | MUA-Au₂₋ₙₘ #2 | NDT-Au₂₋ₙₘ #9 | NDT-Au₂₋ₙₘ #10 |
|---|---|---|---|---|
| 1.5379 | 18.1097 | 4.4274 | 4.8547 | 5.23877 |

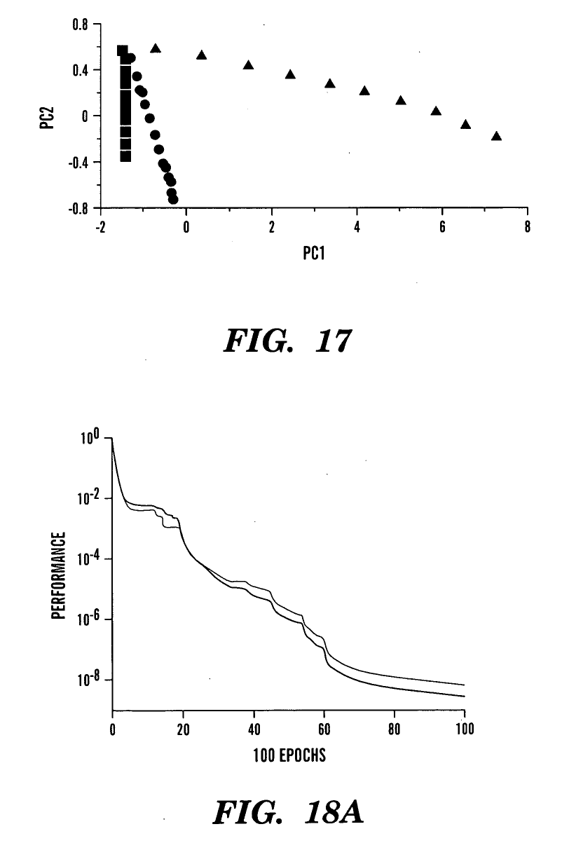

The responses form the selected 5 sensor elements are first preprocessed to eliminate some noise, and then the principal component analysis (PCA) is applied for feature extraction. PCA is a mathematical method that converts a large number of potentially correlated variables into relatively small number of uncorrelated variables. PCA is used in this work for variable dimension reduction (feature extraction) and clustering purpose. The responses of the five-sensor array to mixture (H₂O+acetone), H₂O, and acetone are studied. The score plots in PC1-PC2 plane are shown in FIG. 17. It's observed that each of the three vapors is well separated from the others.

The main PC components obtained by PCA served as inputs to artificial neural networks (ANNs) for pattern recognition. The sensor array performance was examined by performing pattern recognition with a Back Propagation Neural Network (BPN) on the sensor array responses to water and mixture.

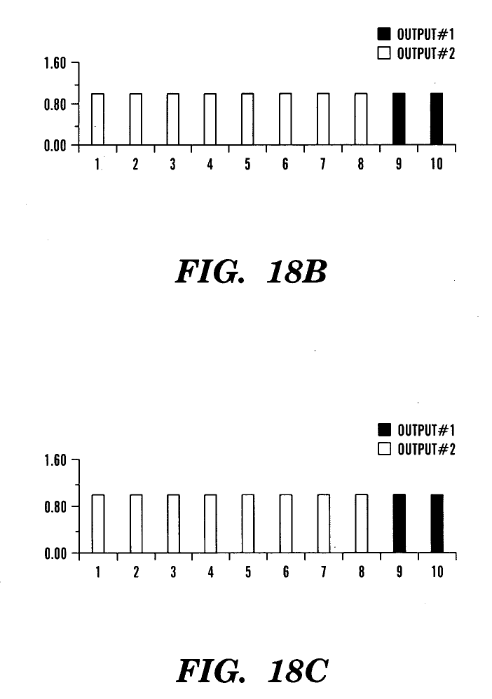

Since the first principle component has explained 97.3% of the variance, PC1 is used as the only input unit of the BPN. There are two units in the output layer of the BPN, in which each unit stands for the presence (+1) or absence (0) of the targeted vapor. The target output for water and water/acetone mixture are (1,0), and (0,1), respectively. The complete data set of 50 response patterns was split into a 30-pattern training set, a 10-pattern verifying set, and a 10-pattern test set. The test set consisted of testing vapors having ten different concentration levels. As shown in FIG. 18A, the performance of the BPN is evaluated by Mean Square Error (MSE), which yields a value of 2.6×10⁻⁹ in this case. The fact that this value is so close to 0 suggests an optimal performance of this BPN. As shown in FIGS. 18B and 18C for the detailed BPN output and corresponding target pattern for each test pattern, the recognition rates for the test vapors are 100%.

Herein has been demonstrated the viability of chemiresistive sensor array consisting of thin film assemblies of metal and alloy nanoparticles that can be tuned by interparticle molecule linkers of different chain lengths for the detection of VOCs. The results have shown that the response sensitivity of the array is dependent on the chain length of the molecular linkers as a result of the change in interparticle spacing. The results have demonstrated a clear dependence of the response sensitivity on the interparticle spacing in the thin film assembly. The most significant change of the response sensitivity vs. chain length was observed to occur in the range of n=9˜13, which is believed to reflect the interparticle spatial effect on the relative change of the thin film conductivity. The relative sensitivity of the vapor sorption induced change in conductivity to interparticle alkyl interactions reflect the important role played by the thermodynamic factors in the nanostructured sensing properties. The change of the sensor array response separation capabilities with the interparticle spacing manipulation is also supported by the statistical analysis results based on ANOVA and Principal Component Analysis. The significance of the interparticle fine-tuning capability of the nanostructured spatial properties is the implication to establishing a detailed delineation between the interparticle spatial properties and the nanostructured sensing materials for the design of chemical sensor arrays.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A detector for acetone comprising:
a sensing platform comprising thin film assemblies of metal or alloy core, ligand-capped nanoparticles and molecular linkers connecting the nanoparticles;
a plurality of transducers mounted on the sensing platforms; and
an artificial neural network operably linked to a voltage source and the plurality of transducers and designed to recognize contact of acetone with the sensing platform.

2. The detector of claim 1, wherein the transducers are quartz-crystal microbalances.

3. The detector of claim 1, wherein the transducers are interdigitated microelectrodes.

4. The detector of claim 1 further comprising a micro controller operably linked to the transducers.

5. The detector of claim 1 further comprising a circuit board operably linked to the transducers.

6. The detector of claim 1, wherein the molecular linkers are selected from the group consisting of α,ω-alkyldithiols, α,ω-dicarboxylic acids, mercaptocarboxylic acids, and combinations thereof.

7. The detector of claim 6, wherein the molecular linkers are α,ω-alkyldithiols.

8. The detector of claim 7, wherein the α,ω-alkyldithiol is HS—(CH₂)ₙ—SH, with n being 3-10.

9. The detector of claim 6, wherein the molecular linkers are α,ω-dicarboxylic acids.

10. The detector of claim 9, wherein the α,ω-dicarboxylic acid is HO₂C—(CH₂)ₙ—CO₂H, with n being 2 to 16.

11. The detector of claim 6, wherein the molecular linkers are mercaptocarboxylic acids.

12. The detector of claim 11, wherein the mercaptocarboxylic acids is HS—(CH₂)ₙ—CO₂H, with n being 2 to 18.

13. The detector of claim 1, wherein the detector comprises a plurality of different sensing platforms.

14. The detector of claim 13, wherein the different sensing platforms differ with regard to the nanoparticle capping ligands, the nanoparticle cores, the molecular linkers, and/or film thickness.

15. The detector of claim 14, wherein the nanoparticle cores differ by size or material.

16. The detector of claim 14, wherein the capping ligands differ by size or material.

17. The detector of claim 14, wherein the molecular linkers differ by length or chemical content.

18. The detector of claim 1, wherein the neural network is trained to distinguish contact of acetone with the sensing platform from contact of other agents with the sensing platform.

19. The detector of claim 1, wherein the neural network is trained to quantitate acetone concentration contacting the sensing platform.

20. The detector of claim 1, wherein the nanoparticle capping ligand is selected from the group consisting of alkanethiols, alkyl amines, alkyl alcohols, alkanoic acids, or mixtures thereof.

21. The detector of claim 20, wherein the nanoparticle capping ligand is decanethiol.

22. The detector of claim 1, wherein the core material of the nanoparticles is selected from the group consisting of gold, silver, platinum, iron oxide, gold-silver alloy, gold-platinum alloy, gold-copper alloy, or mixtures thereof.

23. The detector of claim 22, wherein the core material of the nanoparticles is gold.

24. A method of detecting acetone in a fluid comprising:
providing a fluid and contacting the fluid with the detector of claim 1 under conditions effective to detect acetone in the fluid.

25. The method of claim 24, wherein the fluid is a gas.

26. The method of claim 25, wherein the gas is a breath stream.

27. The method of claim 24, wherein the molecular linkers are selected from the group consisting of α,ω-alkyldithiols, α,ω-dicarboxylic acids, mercaptocarboxylic acids, and combinations thereof.

28. The method of claim 27, wherein the molecular linkers are α,ω-alkyldithiols.

29. The method of claim 28, wherein the α,ω-alkyldithiols is HS—(CH₂)ₙ—SH, with n being 3-10.

30. The method of claim 27, wherein the molecular linkers are α,ω-dicarboxylic acids.

31. The method of claim 30, wherein the α,ω-dicarboxylic acid is HO₂C—(CH₂)ₙ—CO₂H, with n being 2 to 20.

32. The method of claim 27, wherein the molecular linkers are mercaptocarboxylic acids.

33. The method of claim 32, wherein the mercaptocarboxylic acid is HS—(CH₂)ₙ—CO₂H, with n being 2 to 18.

34. The method of claim 24, wherein the detector comprises a plurality of different sensing platforms.

35. The method of claim 34, wherein the different sensing platforms differ with regard to the nanoparticle capping ligands, the nanoparticle cores, the molecular linkers, and/or film thickness.

36. The method of claim 35, wherein the nanoparticle cores differ by size or material.

37. The method of claim 35, wherein the capping ligands differ by size or material.

38. The method of claim 35, wherein the molecular linkers differ by length or chemical content.

39. The method of claim 24 wherein the neural network is trained to distinguish contact of acetone with the sensing platform from contact of other agents with the sensing platform.

40. The method of claim 39, wherein the neural network is trained to quantitate acetone concentration contacting the sensing platform.

41. The method of claim 24, wherein the nanoparticle capping ligand is selected from the group consisting of alkanethiols, alkyl amines, alkyl alcohols, alkanoic acids, or mixtures thereof.

42. The method of claim 41, wherein the nanoparticle capping ligand is decanethiol.

43. The method of claim 24, wherein the core material of the nanoparticles is selected from the group consisting of gold, silver, platinum, iron oxide, gold-silver alloy, gold-platinum alloy, gold-copper alloy, or mixtures thereof.

44. The method of claim 43, wherein the core material of the nanoparticles is gold.
